(19) United States
(12) Patent Application Publication
(10) Pub. No.: US 2008/0015227 A1
(43) Pub. Date: Jan. 17, 2008

(54) Inhibitors of diacylglycerol O-acyltransferase type 1 enzyme

(76) Inventors: Philip R. Kym, Libertyville, IL (US); Gang Zhao, Northbrook, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/749,940

(22) Filed: May 17, 2007

Related U.S. Application Data
(60) Provisional application No. 60/801,838, filed on May 19, 2006.

(51) Int. Cl. C07C 275/28; A61K 31/17; A61K 31/196; A61K 31/215; A61K 31/235; A61K 31/381; A61K 31/44; C07D 333/06; C07D 211/70; A61K 31/454; A61P 1/16; A61P 3/00; A61P 3/10; C07C 229/40

(52) U.S. Cl. 514/326; 514/357; 514/438; 514/532; 514/539; 514/564; 514/598; 546/332; 549/75; 560/19; 562/405; 564/52

(57) ABSTRACT

The present invention relates to compounds of formula (I). 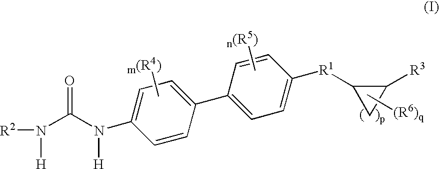
wherein R¹, R², R³, R⁴, R⁵, R⁶, m, n, p and q are defined herein Pharmaceutical compositions and methods for treating DGAT-1 related diseases or conditions are also disclosed.

### CROSS REFERENCE TO RELATED APPLICATIONS

The present application claims the benefit of U.S. Provisional Application No. 60/801,838, filed on May 19, 2006, which is hereby incorporated by reference in its entirety.

### FIELD OF INVENTION

The present invention relates to compounds that are inhibitors of the diacylglycerol O-acyltransferase type 1 (DGAT-1) enzyme Methods of using such compounds to inhibit the activity of diacylglycerol O-acyltransferase type 1 and pharmaceutical compositions including such compounds are also encompassed.

### BACKGROUND OF THE INVENTION

Triacylglycerides represent the major form of energy storage in eukaryotes, and disorders or imbalance in triacylglycerides metabolism are implicated in the pathogenesis and increased risk for obesity, insulin resistance, type II diabetes, nonalcoholic fatty liver disease and coronary heart disease (Lewis, et al., Endocrine Reviews 23:201, 2002). Storage of excess triacylglycerides in lean tissues, such as liver, muscle, and other peripheral tissues, leads to lipid-induced dysfunction in those tissues; thus, reducing fat accumulation in nonadipose sites appears to be of benefit in the treatment of lipotoxicity (Unger, R. H. Endocrinology, 144: 5159-5165, 2003). Accumulation of excess triacylglycerides in white adipose tissue (WAT) leads to obesity, a condition that is associated with decreased life span, type II diabetes, coronary artery disease, hypertension, stroke, and the development of some cancers (Grundy, S. M. Endocrine 13(2): 155-165, 2000). Obesity is a chronic disease that is highly prevalent in modern society and current pharmacological treatment options are limited, creating a need to develop pharmaceutical agents for the treatment of obesity that are safe and effective.

Diacylglycerol O-acyltransferases (DGATs) are membrane-bound enzymes that catalyze the terminal step of triacylglycerides biosynthesis. Two enzymes that display DGAT activity have been characterized: DGAT-1 (diacylglycerol O-acyltransferase type 1) (U.S. Pat. No. 6,100,077; Cases, et al., Proc. Nat. Acad. Sci. 95:13018-13023, 1998) and DGAT-2 (diacylglyerol O-acyltransferase type 2) (Cases, et al., J. Biol. Chem. 276:38870-38876, 2001). DGAT-1 and DGAT-2 share only 12% sequence identity. Significantly, DGAT-1 nill mice are resistant to diet-induced obesity and have increased sensitivity to insulin and leptin (Smith, et al., Nature Genetics 25:87-90, 2000; Chen and Farese. Trends Cardiovasc Med. 10:188, 2000; Chen et al., J. Clin. Invest. 109:10049, 2002). DGAT-1 deficient mice are protected against hepatic steatosis, demonstrate increased energy expenditure, and decreased levels of tissue triacylglycerides. In addition to improved triacylglycerides metabolism, DGAT-1 deficient mice also have improved glucose metabolism, with lower glucose and insulin levels following a glucose load, in comparison to wild-type mice. Partial DGAT-1 deficiency in heterozygous DGAT-1± animals is sufficient to deliver an intermediate phenotype on body weight, adiposity, and insulin and glucose metabolism when compared to wild type and homozygous littermates (Chen and Farese, Arterioscler. Thromb. Vasc. Biol. 25:482-486, 2005), and small molecule DGAT-1 inhibitors have been reported to induce weight loss in diet-induced obese (DIO) mice (US 2004/0224997). The phenotypes of DGAT-1 deficient mice, and the pharmacological activity reported with DGAT-1 inhibitors suggests that the discovery of small molecules that effectively block the conversion of diacylglycerol to triacylglycerides by inhibiting the DGAT-1 enzyme can have utility in the treatment of obesity and other diseases associated with triacylglycerides imbalance.

### SUMMARY OF THE INVENTION

One aspect of the invention is directed towards a compound of formula (I), or a pharmaceutically acceptable salt, prodrug, salt of a prodrug, or a combination thereof, 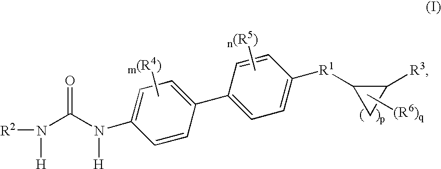
wherein

R¹ is C(O) or C(H)(OH);

R² is alkyl, aryl, heteroaryl or cycloalkyl; wherein each of the aryl, heteroaryl and cycloalkyl is independently unsubstituted or substituted with 1, 2, 3, 4, or 5 substituents independently selected flow the group consisting of alkyl, alkenyl, alkynyl, nitro, —CN, halogen, ethylenedioxy, methylenedioxy, haloalkyl, —OR^{a}, —O—C(O)(R^{a}), —S(R^{a}), —S(O)(R^{b}), —S(O)₂(R^{b}), —C(O)(R^{a}), —C(O)(OR^{a}), —N(R^{a})₂, —N(R^{a})—C(O)(R^{a}), —C(O)N(R^{a})₂, —S(O)₂N(R^{a})₂, R⁷, —(CR^{c}R^{d})ₜ—OR^{a}, —(CR^{c}R^{d})ₜ—O—C(O)(R^{a}), —(CR^{c}R^{d})ₜ—S(R^{a}), —(CR^{c}R^{d})ₜ—S(O)(R^{b}), —(CR^{c}R^{d})ₜ—S(O)₂(R^{b}), —(CR^{c}R^{d})ₜ—C(O)(R^{a}), —(CR^{c}R^{d})ₜ—C(O)(OR^{a}), —(CR^{c}R^{d})ₜ—N(R^{a})₂, —(CR^{c}R^{d})ₜ—N(R^{a})—C(O)(R^{a}), —(CR^{c}R^{d})ₜ—C(O)N(R^{a})₂, —(CR^{c}R^{d})ₜ—S(O)₂N(R^{a})₂ and —(CR^{c}R^{d})ₜ—R⁷;

R³, at each occurrence, is independently —C(O)O(R⁸) or —C(O)N(R⁸)₂;

R⁴ and R⁵ represent substituent groups independently selected from the group consisting of alkyl, alkenyl, alkynyl, nitro, —CN, halogen, haloalkyl, —OR^{e}, —O—C(O)(R^{e}), —S(R^{e}), —S(O)(R^{f}), —S(O)₂(R^{f}), —C(O)(R^{e}), —C(O)(OR^{c}), —N(R^{c})₂, —N(R^{e})—C(O)(R^{e}), —C(O)N(R^{e})₂, —S(O)₂N(R^{e})₂, —(CR^{c}R^{d})ₜ—OR^{e}, —(CR^{c}R^{d})ₜ—O—C(O)(R^{e}), —(CR^{c}R^{d})ₜ—S(R^{e}), —(CR^{c}R^{d})ₜ—S(O)(R^{f}), —(CR^{c}R^{d})ₜ—S(O)₂(R^{f}), —(CR^{c}R^{d})ₜ—C(O)(R^{e}), —(CR^{c}R^{d})ₜ—C(O)(OR^{e}), —(CR^{c}R^{d})ₜ—N(R^{e})₂, —(CR^{c}R^{d})ₜ—N(R^{e})—C(O)(R^{e}), —(CR^{c}R^{d})ₜ—C(O)N(R^{e})₂, and —(CR^{c}R^{d})ₜ—S(O)₂N(R^{e})₂;

R⁶ represents a substituent group selected flow the group consisting of alkyl, OR^{a}, and halogen;

R⁷, at each occurrence, is independently aryl, heteroaryl, cycloalkyl, cycloalkenyl or heterocycle; wherein each R⁷ is independently unsubstituted or substituted with 1, 2, 3, 4 or 5 substituents selected from the group consisting of alkyl, alkenyl, alkynyl, nitro, —CN, halogen, ethylenedioxy, methylenedioxy, haloalkyl, —OR^{e}, —O—C(O)(R^{e}), —S(R^{e}), —S(O)(R^{f}), —S(O)₂(R^{f}), —C(O)(R^{e}), —C(O)(OR^{e}), —N(R^{e})₂, —N(R^{e})—C(O)(R^{e}), —C(O)N(R^{e})₂, —S(O)₂N(R^{e})₂, —(CR^{c}R^{d})ₜ—OR^{e}, —(CR^{c}R^{d})ₜ—O—C(O)(R^{e}), —(CR^{c}R^{d})ₜ—S(R^{e}), —(CR^{c}R^{d})ₜ—S(O)(R^{f}), —(CR^{c}R^{d})ₜ—S(O)₂(R^{f}), —(CR^{c}R^{d})ₜ—C(O)(R^{e}), —(CR^{c}R^{d})ₜ—C(O)(OR^{e}), —(CR^{c}R^{d})ₜ—N(R^{e})₂, —(CR^{c}R^{d})ₜ—N(R^{e})—C(O)(R^{e}), —(CR^{c}R^{d})ₜ—C(O)N(R^{e})₂, and —(CR^{c}R^{d})ₜ—S(O)₂N(R^{e})₂;

R⁸, at each occurrence, is independently hydrogen or alkyl;

R^{a}, at each occurrence, is independently hydrogen, alkyl, haloalkyl, R⁷, or —(CR^{c}R^{d})ₜ—R⁷;

R^{b}, at each occurrence, is independently alkyl, haloalkyl, R⁷, or —(CR^{c}R^{d})ₜ—R⁷;

R^{c}, R^{d} and R^{e}, at each occurrence, are each independently hydrogen, alkyl or haloalkyl;

R^{f}, at each occurrence, is independently alkyl or haloalkyl;

t is 1, 2, 3 or 4;

m is 0, 1, 2 or 3;

n is , 1, 2 or 3;

p is 1, 2, 3, 4 or 5; and

q is 0, 1 or 2.

Another aspect of the invention provides methods of treating various diseases or conditions in a mammal, preferably a human, wherein the methods include administering to the mammal in need thereof a therapeutically effective amount of a compound of the invention as set forth above or a pharmaceutical compositions including a compound of the invention and a pharmaceutically acceptable carriers. In another aspect, the invention provides methods of preventing or treating a disease or condition related to elevated lipid levels, such as plasma lipid levels, especially elevated triacylglycerides levels, in a mammal, especially human, afflicted with such elevated levels, including administering to the mammal a therapeutically or prophylactically effective amount of a compound or a composition as disclosed herein. The invention also relates to novel compounds having therapeutic ability to reduce lipid levels in a mammal, especially triacylglycerides levels In another aspect, the invention provides pharmaceutically compositions including the compound of the invention as set forth above, and a pharmaceutically acceptable carrier. In one embodiment, the present invention relates to methods of treating various conditions in a patient (for example, mammals) including the step of administering to the patient a pharmaceutical composition containing an amount of the compound of the invention that is effective in treating the target condition, and a pharmaceutically acceptable carrier.

### DETAILED DESCRIPTION OF THE INVENTION

For a variable that occurs more than one time in any substituent or in the compound of the invention or any other formulae herein, its definition on each occurrence is independent of its definition at every other occurrence. Combinations of substituents are permissible only if such combinations result in stable compounds. Stable compounds are compounds, which can be isolated in a useful degree of purity from a reaction mixture,

As used in the specification and the appended claims, unless specified to the contrary, the following terms have the meaning indicated:

The term “alkenyl” as used herein, means to a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-meth yl-2-propenyl, 3-butenyl, 4-pentenyl, 5-hexenyl, 2-heptenyl, 2-methyl -1-heptenyl, and 3-decenyl.

The term “alkyl” as used herein, means a straight or branched chain hydrocarbon containing from 1 to 10 carbon atoms. The term “lower alkyl” means a straight or branched chain hydrocarbon containing 1 to 6 carbon atoms Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl

The term “alkynyl” as used herein, means to a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl” as used herein, means phenyl or a bicyclic aryl. The bicyclic aryl is naphthyl, or a phenyl fused to a monocyclic cycloalkyl, or a phenyl fused to a monocyclic cycloalkenyl. The phenyl and the bicyclic aryl groups of the present invention are unsubstituted or substituted. The bicyclic aryl is attached to the parent molecular moiety through any carbon atom contained within the bicyclic aryl. Representative examples of the aryl groups include, but are not limited to, dihydroindenyl, indenyl, naphthyl, dihydronaphthalenyl, 1,3-benzodioxolyl, 2,3-dihydro-1,4-benzodioxin-6-yl, and 5,6,7,8-tetrahydronaphthalenyl

The term “cyano” as used herein means a —CN group.

The term “cycloalkyl” or “cycloalkane” as used herein, means a monocyclic or bicyclic cycloalkyl. The monocyclic cycloalkyl has three to eight carbon atoms, zero heteroatom and zero double bond. Examples of monocyclic ring systems include cyclopropyl, cyclobutyl, cyclopentyl, cyclohexyl, cycloheptyl, and cyclooctyl. The bicyclic cycloalkyl is a monocyclic cycloalkyl fused to a monocyclic cycloalkyl or a monocyclic cycloalkyl in which two non-adjacent carbon atoms of the monocyclic cycloalkyl are linked by an alkylene bridge of one, two or three carbon atoms. Representative examples of bicyclic cycloalkyl include, but are not limited to, adamantyl. The monocyclic and the bicyclic cycloalkyls can be attached to the parent molecular moiety through any substitutable atom contained within the monocyclic and bicyclic cycloalkyls, and can be unsubstituted or substituted

The term “cycloalkenyl” or “cycloalkene” as used herein, means a monocyclic or a bicyclic hydrocarbon ring system. The monocyclic cycloalkenyl has four-, five-, six-, seven- or eight carbon atoms and zero heteroatom. The four-membered ring systems have one double bond, the five-or six-membered ring systems have one or two double bonds, and the seven- or eight-membered ring systems have one, two or three double bonds. Representative examples of monocyclic cycloalkenyl groups include, but not limited to, cyclobutenyl, cyclopentenyl, cyclohexenyl, cycloheptenyl and cyclooctenyl. The bicyclic cycloalkenyl is a monocyclic cycloalkenyl fused to a monocyclic cycloalkyl group, or a monocyclic cycloalkenyl fused to a monocyclic cycloalkenyl group. Representative examples of the bicyclic cycloalkenyl groups include, but not limited to, 4,5,6,7-tetrahydro-3aH-indenie, octahydronaphthalenyl and 1,6-dihydropentalene, The monocyclic and bicyclic cycloalkenyl groups of the present invention can be attached to the parent molecular moiety through any substitutable atom contained within the monocyclic and the bicyclic cycloalkenyls, and can be unsubstituted or substituted.

The term “ethylenedioxy” as used herein, means a —O—(CH₂)₂—O— group wherein the oxygen atoms of the ethylenedioxy group are attached to two adjacent carbon atoms of a phenyl or naphthyl moiety, forming a six membered ring with the phenyl or naphthyl moiety that it is attached to.

The term “halo” or “halogen” as used herein, means —Cl, —Br, —I or —F.

The term “haloalkyl” as used herein, means an alkyl group, as defined herein, in which one, two, three, four, five or six hydrogen atoms are replaced by halogen Representative examples of haloalkyl include, but are not limited to, chloromethyl, 2-fluoroethyl, trifluoromethyl, pentafluoroethyl, and 2-chloro-3-fluoropentyl.

The term “heterocycle” or “heterocyclic” as used herein, means a monocyclic heterocycle, or a bicyclic heterocycle. The monocyclic heterocycle is a three-, four-, five-, six- or seven-membered ring containing at least one heteroatom independently selected from the group consisting of O, N, and S. The three- or four-membered ring contains zero or one double bond, and one heteroatom selected from the group consisting of O, N and S. The five-membered ring contains zero or one double bond and one, two or three heteroatoms selected from the group consisting of O, N and S. Tile six-membered ring contains zero, one or two double bonds and one, two or three heteroatoms selected from the group consisting of O, N and S The seven-membered ring contains zero, one, two, or three double bonds and one, two or three heteroatoms selected from the group consisting of O, N and S. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, a monocyclic heterocycle fused to a monocyclic heterocycle. Representative examples of bicyclic heterocycle include, but are not limited to, 1,3-benzodithiolyl, benzopyranyl, benzothiopyranyl, 2,3-dihydrobenzofuranyl, 2,3-dihydrobenzothienyl, 2,3-dihydro-1H-indolyl, 2,3-dihydroisoindol-2-yl, 2,3-dihydroisoindol-3-yl, 1,3-dioxo-1H-isoindolyl, 2-(trifluoromethyl)-5,6-dihydroimidazo-[1,2-a]pyrazin-7(8H)-yl, 1-acetyl-2,3-dihydro-1H-indol-6-yl, 3-(trifluoromethyl)-5,6-dihydro[1,2,4]triazolo[4,3-a]pyrazin-7(8H)-yl, 1,2,3,4-tetrahydroisoquinolin-2-yl, and 1,2,3,4-tetrahydroquinolinyl The monocyclic and bicyclic heterocycles of the present invention can be connected to the parent molecular moiety through any carbon atom or any nitrogen atom contained within the monocyclic and the bicyclic heterocycles, and can be unsubstituted or substituted.

The term “heteroaryl” as used herein, means a monocyclic heteroaryl, or a bicyclic heteroaryl. The monocyclic heteroaryl is a five- or six-membered ring. The five-membered ring contains two double bonds, and one, two, three or four nitrogen atoms; or one or two nitrogen atoms and optionally one oxygen or sulfur atom. The six-membered ring contains three double bonds and one, two, three or four nitrogen atoms. Representative examples of monocyclic heteroaryl include, but are not limited to, furanyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyt, thiazolyl, thienyl, triazolyl, and triazinyl. The bicyclic heteroaryl consists of a monocyclic heteroaryl fused to a phenyl, or a monocyclic heteoaryl fused to a monocyclic cycloalkyl, or a monocyclic heteroaryl fused to a monocyclic cycloalkenyl, or a monocyclic heteroaryl fused to a monocyclic heteroaryl, or a monocyclic heteroaryl fused to a monocyclic heterocycle. Representative examples of bicyclic heteroaryl groups include, but not limited to, benzofuranyl, benzothienyl, benzoxazolyl, benzimidazolyl, benzodioxolyl, 6,7-dihydro-1,3-benzothiazolyl, imidazo[1,2-a]pyridinyl, indazolyl, indolyl, isoindolyl, isoquinolinyl, naphthyridinyl, pyridoimidazolyl, quinolinyl, thiazolo[5,4-b]pyridin-2-yl, thiazolo[5,4-d]pyrimidin-2-yl, and 5,6,7,8-tetrahydroquinolin-5-yl. The monocyclic and the bicyclic heteroaryls are connected to the parent molecular moiety through any carbon atom or any nitrogen atom contained within the monocyclic and the bicyclic heteroaryls, and can be unsubstituted or substituted.

The term “heteroatom” as used herein, refers to nitrogen, oxygen or sulfur atom

The term “methylenedioxy” as used herein, means a —O—(CH₂)—O— group wherein the oxygen atoms of the methylenedioxy group are attached to two adjacent carbon atoms of the phenyl or naphthyl ring, forming a five membered ring with the phenyl or naphthyl moiety that it is attached to.

The term “nitro” as used herein, refers to an —NO₂ group.

“Mammal” includes humans and domestic animals, such as cats, dogs, swine, cattle, horses, and the like.

Compounds of the invention can have the formula (I) as described above,

In compounds of formula (I), R¹ is C(O) or C(H)(OH);

R² is alkyl, aryl, heteroaryl or cycloalkyl; wherein each of the aryl, heteroaryl and cycloalkyl is independently unsubstituted or substituted with substituents as described in the summary of the invention. Particularly, R² is C₁₋₆ alkyl, phenyl, thienyl, pyridinyl, cyclopentyl, cyclohexyl, cycloheptyl, cyclooctyl, adamantyl or 1,2,3,4-tetrahydronaphthalen-1-yl, wherein each R², other than when R² is C₁₋₆ alkyl, is independently unsubstituted or substituted with substituents as described in the summary of the invention;

R³ is —C(O)O(R⁸) or —C(O)N(R⁸)₂ wherein R⁸, at each occurrence, is independently hydrogen or alkyl. Particularly, R⁸ is hydrogen or C₁₋₆ alkyl. More particularly, R⁸ is hydrogen or methyl;

R⁴ and R⁵ represent substituent groups independently selected from the group consisting of alkyl, alkenyl, alkynyl, nitro, —CN, halogen, haloalkyl, —OR^{e}, —O—C(O)(R^{e}), —S(R^{e}), —S(O)(R^{f}), —S(O)₂(R^{f}), —C(O)(R^{e}), —C(O)(OR^{e}), —N(R^{e})₂, —N(R^{e})—C(O)(R^{e}), —C(O)N(R^{e})₂, —S(O)₂N(R^{e})₂, —(CR^{c}R^{d})ₜ—OR^{e}, —(CR^{c}R^{d})ₜ—O—C(O)(R^{e}), —(CR^{c}R^{d})ₜ—S(R^{e}), —(CR^{c}R^{d})ₜ—S(O)(R^{f}), —(CR^{c}R^{d})ₜ—S(O)₂(R^{f}), —(CR^{c}R^{d})ₜ—C(O)(R^{e}), —(CR^{c}R^{d})ₜ—C(O)(OR^{e}), —(R^{c}R^{d})ₜ—N(R^{e})₂, —(CR^{c}R^{d})ₜ—N(R^{e})—C(O)(R^{e}), —(CR^{c}R^{d})ₜ—C(O)N(R^{e})₂, and —(CR^{c}R^{d})ₜ—S(O)₂N(R^{e})₂; wherein R_{c}, R_{d}, Rₑ, R_{f}, and t are as set forth in the summary of the invention;

R⁶ represents a substituent group selected from the group consisting of alkyl, OR^{a}, and halogen;

m is 0, 1, 2 or 3;

n is 0, 1, 2 or 3;

p is 1, 2, 3, 4 or 5; particularly, p is 3 or 4; and

q is 0, 1 or 2; particularly q is 0.

It is appreciated that the present invention contemplates compounds of formula (I) with combinations of the above embodiments, including particular, more particular and most preferred embodiments.

Accordingly, one aspect of the invention relates to a compound of formula (I), or a pharmaceutically acceptable salt, prodrug, salt of a prodrug, or a combination thereof, wherein p is 3, i.e. a compound having the following formula (Ia):

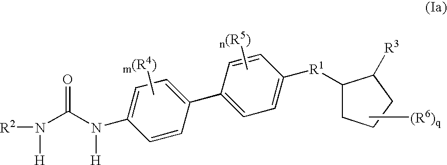

wherein R¹, R², R³, R⁴, R⁵, R⁶, m, n, and q are as described in formula (I). It is understood that embodiments of R¹, R², R³, R⁴, R⁵ and R⁶, and combinations of embodiments, including particular, more particular and most preferred embodiments as described in formula (I) are also contemplated for compounds of formula (Ia).

Of this group of compounds, a subgroup of compounds include those wherein R¹ is C(O); R² is alkyl, particularly. C₁₋₆ alkyl, and more particularly isopropyl, R³ is —C(O)O(R⁸) or —C(O)N(R⁸)₂ wherein R⁸ is hydrogen or alkyl, preferably, R⁸ is hydrogen or C₁₋₆ alkyl, and more preferably R⁸ is hydrogen or methyl, and R⁴, R⁵, R⁶, m, n, and q are as defined in the summary of the invention. Particularly, this subgroup of compounds include those wherein R¹ is C(O); R² is C₁₋₆ alkyl, R³ is —C(O)O(R⁸) or —C(O)N(R⁸)₂ wherein R⁸ is hydrogen or C₁₋₆ alkyl, and R⁴, R⁵, R⁶, m, n, and q are as defined in the summary of the invention. More particularly, this subgroup of compounds include those wherein R¹ is C(O); R² is isopropyl, R³ is —C(O)O(R⁸) or —C(O)N(R⁸)₂ wherein R⁸ is hydrogen or methyl, and R⁴, R⁵, R⁶, m, n, and q are as defined in the summary of the invention.

Of this subgroup of compounds, a set of compounds include those wherein m, n and q are 0.

Of the compounds of formula (Ia), another subgroup of compounds include those wherein R¹ is C(O), R² is aryl, unsubstituted or substituted as set forth in the summary of the invention, particularly, R² is unsubstituted or substituted phenyl; R³ is —C(O)O(R⁸) or —C(O)N(R⁸)₂ wherein R⁸ is hydrogen or alkyl, preferably, R⁸ is hydrogen or C₁₋₆ alkyl, and mole preferably R⁸ is hydrogen or methyl, and R⁴, R⁵, R⁶, m, n, and q are as defined in the summary of the invention. Example of this subgroup include compounds wherein R¹ is C(O), R² is unsubstituted or substituted phenyl, R³ is —C(O)O(R⁸) or —C(O)N(R⁸)₂ wherein R⁸ is hydrogen or C₁₋₆ alkyl, and R⁴, R⁵, R⁶, m, n, and q are as defined in the summary of the invention. More particularly, this subgroup also includes compounds wherein R¹ is C(O), R² is unsubstituted or substituted phenyl, R³ is —C(O)O(R⁸) or —C(O)N(R⁸)₂ wherein R⁸ is hydrogen or methyl, and R⁴, R⁵, R⁶, m, n, and q are as defined in the summary of the invention.

Of this subgroup of compounds, a set of compounds include those wherein m, n and q are 0.

Of the compounds of formula (Ia), another subgroup of compounds include those wherein R¹ is C(H)(OH), R² is aryl, unsubstituted or substituted as set forth in the summary of the invention, particularly, R² is unsubstituted or substituted phenyl; R³ is —C(O)O(R⁸) or —C(O)N(R⁸)₂ wherein R⁸ is hydrogen or alkyl, preferably, R⁸ is hydrogen or C₁₋₆ allyl, and more preferably R⁸ is hydrogen or methyl, and R⁴, R⁵, R⁶, m, n, and q are as defined in the summary of the invention. Example of this subgroup include compounds wherein R¹ is C(H)(OH), R² is unsubstituted or substituted phenyl, R³ is —C(O)O(R⁸) or —C(O)N(R⁸)₂ wherein R⁸ is hydrogen or C₁₋₆ alkyl, and R⁴, R⁵, R⁶, m, n, and q are as defined in the summary of the invention More particularly, this subgroup also includes compounds wherein R¹ is C(H)(OH), R² is unsubstituted or substituted phenyl, R³ is —C(O)O(R⁸) or —C(O)N(R⁸)₂ wherein R⁸ is hydrogen or methyl, and R⁴, R⁵, R⁶, m, n, and q are as defined in the summary of the invention.

Of this subgroup of compounds, a set of compounds include those wherein m, n and q are 0.

Of the compounds of formula (Ia), another subgroup of compounds include those wherein R¹ is C(O), R² is heteroaryl, unsubstituted or substituted as set forth in the summary of the invention, particularly, R² is thienyl or pyridinyl, each of which is independently unsubstituted or substituted as set forth in the summary of the invention, R³ is —C(O)O(R⁸) or —C(O)N(R⁸)₂ wherein R⁸ is hydrogen or alkyl, preferably, R⁸ is hydrogen or C₁₋₆ alkyl, and more preferably R⁸ is hydrogen or methyl, and R⁴, R⁵, R⁶, m, n, and q are as defined in the summary of the invention. Example of this subgroup include compounds wherein R¹ is C(O), R² is thienyl or pyridinyl, each of which is unsubstituted or substituted, R³ is —C(O)O(R⁸) or —C(O)N(R⁸)₂ wherein R⁸ is hydrogen or C₁₋₆ alkyl, and R⁴, R⁵, R⁶, m, n, and q are as defined in the summary of the invention. More particularly, this subgroup also includes compounds wherein R¹ is C(O), R² is thienyl or pyridinyl, each of which is unsubstituted or substituted, R³ is —C(O)O(R⁸) or —C(O)N(R⁸)₂ wherein R⁸ is hydrogen or methyl, and R⁴, R⁵, R⁶, m, n, and q are as defined in the summary of the invention.

Of this subgroup of compounds, a set of compounds include those wherein m, n and q are 0.

Of the compounds of formula (Ia), yet another subgroup of compounds include those wherein R¹ is C(O), R² is cycloalkyl, unsubstituted or substituted as set forth in the summary of the invention, particularly, R² is cyclopentyl, cyclohexyl, cycloheptyl, cyclooctyl, admantyl or 1,2,3,4-tetrahydronaphthalen-1-yl, each of which is independently unsubstituted or substituted as set forth in the summary of the invention; R³ is —C(O)O(R⁸) or —C(O)N(R⁸)₂ wherein R⁸ is hydrogen or alkyl, preferably, R⁸ is hydrogen or C₁₋₆ alkyl, and more preferably R⁸ is hydrogen or methyl, and R⁴, R⁵, R⁶, m, n, and q are as defined in the summary of the invention. Example of this subgroup include compounds wherein R¹ is C(O), R² is cyclopentyl, cyclohexyl, cycloheptyl, cyclooctyl, admantyl or 1,2,3,4-tetrahydronaphthalen-1-yl, each of which is unsubstituted or substituted, R³ is —C(O)O(R⁸) or —C(O)N(R⁸)₂ wherein R⁸ is hydrogen or C₁₋₆ alkyl, and R⁴, R⁵, R⁶, m, n, and q are as defined in the summary of the invention. More particularly, this subgroup also includes compounds wherein R¹ is C(O), R² is cyclopentyl, cyclohexyl, cycloheptyl, cyclooctyl, admantyl or 1,2,3,4-tetrahydronaphthalen-1-yl, each of which is unsubstituted or substituted, R³ is —C(O)O(R⁸) or —C(O)N(R⁸)₂ wherein R⁸ is hydrogen or methyl, and R⁴, R⁵, R⁶, m, n, and q are as defined in the summary of the inventions

Of this subgroup of compounds, a set of compounds include those wherein m, n and q are 0.

Another aspect of the invention relates to a compound of formula (I), or a pharmaceutically acceptable salt, prodrug, salt of a prodrug, or a combination thereof wherein p is 4, i.e a compound having the following formula (Ib):

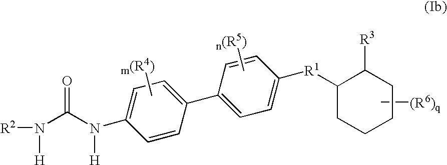

wherein R¹, R², R³, R⁴, R⁵, R⁶, m, n, and q are as described in formula (I). It is understood that embodiments of R¹, R², R³, R⁴, R⁵ and R⁶, and combinations of embodiments, including particular, more particular and most preferred embodiments as described in formula (I) are also contemplated for compounds of formula (Ib),

Of this group of compounds, there is a subgroup of compounds wherein R¹ is C(O), R² is aryl, unsubstituted or substituted as set forth in the summary of the invention, particularly, R² is unsubstituted or substituted phenyl; R³ is —C(O)O(R⁸) or —C(O)N(R⁸)₂ wherein R⁸ is hydrogen or alkyl, preferably, R⁸ is hydrogen or C₁₋₆ alkyl, and more preferably R⁸ is hydrogen or methyl, and R⁴, R⁵, R⁶, m, n, and q are as defined in the summary of the invention. Examples of this subgroup include compounds wherein R¹ is C(O), R² is unsubstituted or substituted phenyl, R³ is —C(O)O(R⁸) or —C(O)N(R⁸)₂ wherein R⁸ is hydrogen or C₁₋₆ alkyl, and R⁴, R⁵, R⁶, m, n, and q are as defined in the summary of the invention. More particularly, this subgroup also includes compounds wherein R¹ is C(O), R² is unsubstituted or substituted phenyl, R³ is —C(O)O(R⁸) or —C(O)N(R⁸ )₂ wherein R⁸ is hydrogen or methyl, and R⁴, R⁵, R⁶, m, n, and q are as defined in the summary of the invention.

Of this subgroup of compounds, a set of compounds include those wherein m, n and q are 0.

Exemplary compounds of the present invention include, but are not limited to the following:

methyl (1R,2R)-2-({4′-[({[4-(trifluoromethoxy)phenyl]amino}carbonyl)amino]-1,1′-biphenyl-4-yl}carbonyl)cyclopentanecarboxylate;

(1R,2R)-2-({4′-[({[4-(trifluoromethoxy)phenyl]amino}carbonyl)amino]-1,1′-biphenyl-4-yl}carbonyl)cyclopentanecarboxylic acid;

methyl (1R,2R)-2-({4′-[({[4-(trifluoromethyl)phenyl]amino}carbonyl)amino]-1,1′-biphenyl-4-yl}carbonyl)cyclopentanecarboxylate;

(1R,2R)-2-({4′-[({[4-(trifluoromethyl)phenyl]amino}carbonyl)amino]-1,1′-biphenyl-4-yl}carbonyl)cyclopentanecarboxylic acid;

methyl (1R,2R)-2-[(4′-{[(1,3-benzodioxol-5-ylamino)carbonyl]amino}-1,1′-biphenyl-4-yl)carbonyl]cyclopentanecarboxylate;

(1R,2R)-2-[(4′-{[(1,3-benzodioxol-5-ylamino)carbonyl]amino}-1,1′-biphenyl-4-yl)carbonyl]cyclopentanecarboxylic acid;

methyl (1R,2R)-2-{[4′-({[(4-acetylphenyl)amino]carbonyl}amino)-1,1′-biphenyl-4-yl]carbonyl}cyclopentanecarboxylate;

(1R,2R)-2-{[4′-({[(4-acetylphenyl)amino]carbonyl}amino)-1,1′-biphenyl-4-yl]carbonyl}cyclopentanecarboxylic acid;

methyl (1R,2R)-2-({4′-[(anilinocarbonyl)amino]-1,1′-biphenyl-4-yl}carbonyl)cyclopentanecarboxylate;

(1R,2R)-2-({4′-[(anilinocarbonyl)amino]-1,1′-biphenyl-4-yl}carbonyl)cyclopentanecarboxylic acid;

methyl (1R,2R)-2-{[4′-({[(2-chlorophenyl)amino]carbonyl}amino)-1,1′-biphenyl-4-yl]carbonyl}cyclopentanecarboxylate;

(1R,2R)-2-{[4′-({[(2-chlorophenyl)amino]carbonyl}amino)-1,1′-biphenyl-4-yl]carbonyl}cyclopentanecarboxylic acid;

methyl (1R,2R)-2-{[4′-({[(3-chlorophenyl)amino]carbonyl}amino)-1,1′-biphenyl-4-yl]carbonyl}cyclopentanecarboxylate;

(1R,2R)-2-{[4′-({[(3-chlorophenyl)amino]carbonyl}amino)-1,1′-biphenyl-4-yl]carbonyl}cyclopentanecarboxylic acid;

methyl (1R,2R)-2-{[4′-({[(4-chlorophenyl)amino]carbonyl}amino)-1,1′-biphenyl-4-yl]carbonyl}cyclopentanecarboxylate;

(1R,2R)-2-{[4′-({[(4-chlorophenyl)amino]carbonyl}amino)-1,1′-biphenyl-4-yl]carbonyl}cyclopentanecarboxylic acid;

methyl (1R,2R)-2-{[4′-({[(4-cyanophenyl)amino]carbonyl}amino)-1,1′-biphenyl-4-yl]carbonyl}cyclopentanecarboxylate;

(1R,2R)-2-{[4′-({[(4-cyanophenyl)amino]carbonyl}amino)-1,1′-biphenyl-4-yl]carbonyl}cyclopentanecarboxylic acid;

methyl (1R,2R)-2-{[4′-({[(4-methoxyphenyl)amino]carbonyl}amino)-1,1′-biphenyl-4-yl]carbonyl}cyclopentanecarboxylate;

(1R,2R)-2-{[4′-({[(4-methoxyphenyl)amino]carbonyl}amino)-1,1′-biphenyl-4-yl]carbonyl cyclopentanecarboxylic acid;

methyl (1R,2R)-2-[(4′-{[(isopropylamino)carbonyl]amino}-1,1′-biphenyl-4-yl)carbonyl]cyclopentanecarboxylate;

(1R,2R)-2-[(4′-{[(isopropylamino)carbonyl]amino}-1,1′-biphenyl-4-yl)carbonyl]cyclopentanecarboxylic acid;

methyl 2-({4′-[({[4-(trifluoromethyl)phenyl]amino}carbonyl)amino]-1,1′-biphenyl-4-yl}carbonyl)cyclohexanecarboxylate;

2-({4′-[({[4-(trifluoromethyl)phenyl]amino}carbonyl)amino]-1,1′-biphenyl-4-yl}carbonyl)cyclohexanecarboxylic acid;

methyl 2-{[4′-({[(3-chlorophenyl)amino]carbonyl}amino)-1,1′-biphenyl-4-yl]carbonyl}cyclohexanecarboxylate;

2-{[4′-({[(3-chlorophenyl)amino]carbonyl}amino)-1,1′-biphenyl-4-yl]carbonyl}cyclohexanecarboxylic acid;

(1R,2R)-2-((R)-hydroxy{4′-[({[4-(trifluoromethyl)phenyl]amino}carbonyl)amino]-1,1′-biphenyl-4-yl}methyl)cyclopentanecarboxylic acid;

(1R,2R)-2-((S)-hydroxy{4′-[({[4-(trifluoromethyl)phenyl]amino}carbonyl)amino]-1,1′-biphenyl-4-yl}methyl)cyclopentanecarboxylic acid;

methyl (1R,2R)-2-{[4′-({[(4-fluorophenyl)amino]carbonyl}amino)-1,1′-biphenyl-4-yl]carbonyl}cyclopentanecarboxylate;

(1R,2R)-2-{[4′-({[(4-fluorophenyl)amino]carbonyl}amino)-1,1′-biphenyl-4-yl}carbonyl}cyclopentanecarboxylic acid;

methyl (1R,2R)-2-{[4′-({[(3-fluorophenyl)amino]carbonyl}amino)-1,1′-biphenyl-4-yl]carbonyl}cyclopentanecarboxylate;

(1R,2R)-2-{[4′-({[(3-fluorophenyl)amino]carbonyl}amino)-1,1′-biphenyl-4-yl]carbonyl}cyclopentanecarboxylic acid;

methyl (1R,2R)-2-{[4′-({[(2-fluorophenyl)amino]carbonyl}amino)-1,1′-biphenyl-4-yl]carbonyl}cyclopentanecarboxylate;

(1R,2R)-2-{[4′-({[(2-fluorophenyl)amino]carbonyl}amino)-1,1′-biphenyl-4-yl]carbonyl}cyclopentanecarboxylic acid;

methyl 2-{[4′-({[(4-fluorophenyl)amino]carbonyl}amino)-1,1′-biphenyl-4-yl]carbonyl}cyclohexanecarboxylate;

2-{[4′-({[(4-fluorophenyl)amino]carbonyl}amino)-1,1′-biphenyl-4-yl]carbonyl}cyclohexanecarboxylic acid;

methyl (1R,2R)-2-({4′-[({[3-(trifluoromethyl)phenyl]amino}carbonyl)amino]-1,1′-biphenyl-4-yl}carbonyl)cyclopentanecarboxylate;

(1R,2R)-2-({4′-[({[3-(trifluoromethyl)phenyl]amino}carbonyl)amino]-1,1′-biphenyl-4-yl}carbonyl)cyclopentanecarboxylic acid;

methyl 2-({4′-[({[3-(trifluoromethyl)phenyl]amino}carbonyl)amino]-1,1′-biphenyl-4-yl}carbonyl)cyclohexanecarboxylate; and

2-({4′-[({[3-(trifluoromethyl)phenyl]amino}carbonyl)amino]-1,1′-biphenyl-4-yl}carbonyl)cyclohexanecarboxylic acid;

methyl (1R,2R)-2-({4′-[({[2-(trifluoromethyl)phenyl]amino}carbonyl)amino]-1,1′-biphenyl-4-yl}carbonyl)cyclopentanecarboxylate;

(1R,2R)-2-({4′-[({[2-(trifluoromethyl)phenyl]amino}carbonyl)amino]-1,1′-biphenyl-4-yl}carbonyl)cyclopentanecarboxylic acid;

methyl 2-({4′-[({[2-(trifluoromethyl)phenyl]amino}carbonyl)amino]-1,1′-biphenyl-4-yl}carbonyl)cyclohexanecarboxylate 2-({4′-[({[2-(trifluoromethyl)phenyl]amino}carbonyl])amino}-1,1′-biphenyl-4-yl}carbonyl)cyclohexanecarboxylic acid; (1R,2R)-2-({4′-[(anilinocarbonyl)amino]-1,1′-biphenyl-4-yl}carbonyl)cyclopentanecarboxamide;

(1R,2R)-2-({4′-[(anilinocarbonyl)amino]-1,1′-biphenyl-4-yl}carbonyl)-N-methylcyclopentanecarboxamide;

methyl (1R,2R)-2-{[4′-({[(2,6-diisopropylphenyl)amino]carbonyl}amino)-1,1′-biphenyl-4-yl]carbonyl}cyclopentanecarboxylate;

(1R,2R)-2-{[4′-({[(2,6-diisopropylphenyl)amino]carbonyl}amino)-1,1′-biphenyl-4-yl]carbonyl}cyclopentanecarboxylic acid;

methyl (1R,2R)-2-{[4′-({[(2,6-dimethylphenyl)amino]carbonyl}amino)-1,1′-biphenyl-4-yl]carbonyl}cyclopentanecarboxylate;

(1R,2R)-2-{[4′-({[(2,6-dimethylphenyl)amino]carbonyl}amino)-1,1′-biphenyl-4-yl]carbonyl}cyclopentanecarboxylic acid;

methyl (1R,2R)-2-({4′-[({[2-fluoro-5-(trifluoromethyl)phenyl]amino}carbonyl)amino]-1,1′-biphenyl-4-yl}carbonyl)cyclopentanecarboxylate;

(1R,2R)-2-({4′-[({[2-fluoro-5-(trifluoromethyl)phenyl]amino}carbonyl)amino]-1,1′-biphenyl-4-yl}carbonyl)cyclopentanecarboxylic acid;

methyl (1R,2R)-2-[(4′-{[(thien-2-ylamino)carbonyl]amino}-1,1′-biphenyl-4-yl)carbonyl]cyclopentanecarboxylate;

(1R,2R)-2-[(4′-{[(thien-2-ylamino)carbonyl]amino}-1,1′-biphenyl-4-yl)carbonyl]cyclopentanecarboxylic acid;

methyl (1R,2R)-2-[(4′-{[(pyridin-3-ylamino)carbonyl]amino}-1,1′-biphenyl-4-yl)carbonyl]cyclopentanecarboxylate;

(1R,2R)-2-[(4′-{[(pyridin-3-ylamino)carbonyl]amino}-1,1′-biphenyl-4-yl)carbonyl]cyclopentanecarboxylic acid;

methyl (1R,2R)-2-[(4′-{[(cyclohexylamino)carbonyl]amino}-1,1′-biphenyl-4-yl)carbonyl]cyclopentanecarboxylate;

(1R,2R)-2-[(4′-{[(cyclohexylamino)carbonyl]amino}-1,1′-biphenyl-4-yl)carbonyl]cyclopentanecarboxylic acid;

methyl (1R,2R)-2-[(4′-{[(1-adamantylamino)carbonyl]amino}-1,1′-biphenyl-4-yl)carbonyl]cyclopentanecarboxylate;

(1R,2R)-2-[(4′-{[(1-adamantylamino)carbonyl]amino}-1,1′-biphenyl-4-yl)carbonyl]cyclopentanecarboxylic acid;

methyl (1R,2R)-2-[(4′-{[(cyclopentylamino)carbonyl]amino}-1,1′-biphenyl-4-yl)carbonyl]cyclopentanecarboxylate;

methyl (1R,2R)-2-[(4′-{[(cycloheptylamino)carbonyl]amino}-1,1′-biphenyl-4-yl)carbonyl]cyclopentanecarboxylate;

methyl (1R,2R)-2-[(4′-{[(1,2,3,4-tetrahydronaphthalen-1-ylamino)carbonyl]amino}-1,1′-biphenyl-4-yl)carbonyl]cyclopentanecarboxylate;

(1R,2R)-2-[(4′-{[(cyclopentylamino)carbonyl]amino}-1,1′-biphenyl-4-yl)carbonyl]cyclopentanecarboxylic acid;

(1R,2R)-2-[(4′-{[(cycloheptylamino)carbonyl]amino}-1,1′-biphenyl-4-yl)carbonyl]cyclopentanecarboxylic acid;

(1R,2R)-2-[(4′-{[(1,2,3,4-tetrahydronaphthalen-1-ylamino)carbonyl]amino}-1,1′-biphenyl-4-yl)carbonyl]cyclopentanecarboxylic acid;

methyl (1R,2R)-2-[(4′-{[(cyclooctylamino)carbonyl]amino}-1,1′-biphenyl-4-yl)carbonyl]cyclopentanecarboxylate; and

(1R,2R)-2-[(4′-{[(cyclooctylamino)carbonyl]amino)}-1,1′-biphenyl-4-yl)carbonyl]cyclopentanecarboxylic acid;

or a pharmaceutically acceptable salt, prodrug, or salt of a prodrug thereof.

The present compounds can exist as pharmaceutically acceptable salts and include both acid and base addition salts. The term “pharmaceutically acceptable” refers to salts or zwitterions of the compounds which are water or oil-soluble or dispersible, suitable for treatment of disorders without undue toxicity, irritation, and allergic response, commensurate with a reasonable benefit/risk ratio, and effective for their intended use The acid addition salts can be prepared during the final isolation and purification of the compounds or separately by reacting an amino group of the compounds with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, L-malate, D-malate, methanesulfonate, naphthylenesulfonate, nicotinate, oxalate, pamoate, pectinate, persulfate, 3-phenylpropionate, picrate, oxalate, pivalate, propionate, succinate, L-tartrate, D-tartrate, trichloroacetic, trifluoroacetic, glutamate, para-toluenesulfonate, undecanoate, hydrochloric, hydrobromic, sulfuric, phosphoric, and the like. The amino groups of the compounds can also be quaternized with alkyl chlorides, bromides, and iodides such as methyl, ethyl, propyl, isopropyl, butyl, lauryl, myristyl, stearyl, and the like.

Basic addition salts can be prepared during the final isolation and purification of the present compounds or by addition of an inorganic or an organic base to the free acid. Salts derived from inorganic bases include, but are not limited to, the ammonium, lithium, sodium, potassium, calcium, iron, zinc, copper, manganese, magnesium, aluminum salts and the like. Preferred inorganic salts are the ammonium, sodium, potassium, calcium and magnesium salts. Salts derived from organic bases include, but are not limited to, salts of primary, secondary, and tertiary amines, substituted amines including naturally occurring substituted amines, and cyclic amines such as ammonia, methylamine, isopropylamine, dimethylamine, trimethylamine, triethylamine, diethylamine, ethylamine, tributylamine, tripropylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethlylenediamine, ethylenediamine, ethanolamine, 2-dimethylaminoethanol, 2-diethylaminoethanol, diethanolamine, lysine, arginine, histidine, procaine, glucosamine, methylglucosamine, ethylenediamine, purines, choline, triethanolamine, piperidine, N-ethylpiperidine, piperazine, and the like, are contemplated as being within the scope of the present invention.

The present compounds can also exist as therapeutically suitable prodrugs. The term “therapeutically suitable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds that are rapidly transformed in vivo to the parent compounds of formula (I) for example, by hydrolysis in blood.

The present compounds can exhibit the phenomena of tautomerism or structural isomerism As the drawings within this specification can only represent one possible tautomeric or structural isomeric form, it should be understood that the invention encompasses any tautomeric or structural isomeric form, or mixtures thereof, which possess the ability to inhabit DGAT-1, and is not limited to any one tautomeric or structural isomeric form utilized within the drawings.

Compounds including geometric isomers of carbon-carbon double bonds and carbon-nitrogen double are meant to be included in this invention. Substituents around a carbon-carbon or a carbon-nitrogen double bond are designated as being of Z or E configuration and substituents around a cycloalkyl or heterocycloalkyl are designated as being of cis or trans configuration.

It will be appreciated by those skilled in the art that the compounds of this invention, exemplified by formula (I) can exist as geometric isomers. All geometric isomeric forms and mixtures thereof of the compounds described herein are intended to be encompassed within the scope of the present invention.

Asymmetric centers exist in the present compounds. Individual stereoisomers of the compounds are prepared by synthesis from chiral starting materials or by preparation of racemic mixtures and separation by conversion to a mixture of diastereomers followed by separation or recrystallization, chromatographic techniques, or direct separation of the enantiomers on chiral chromatographic columns. Starting materials of particular stereochemistry are either commercially available or are made by the methods described herein and resolved by techniques well known in the art.

Compounds of this invention contain at least one chiral center and can exist as single stereoisomers (e.g. single enantiomer), mixtures of stereoisomers (egg any mixture of enantiomers or diastereomers) or racemic mixtures thereof As a result, all stereoisomers of the compounds of the invention are meant to be included in the invention, including racemic mixtures, mixtures of diastereomers, mixtures of enantiomers, as well as individual optical isomers, including, enantiomers and single diastereomers of the compounds of the invention substantially free from their enantiomers or other diastereomers. By “substantially free” is meant greater than about 80% flee of other enantiomers or diastereomers of the compound, more preferably greater than about 90% flee of other enantiomelrs or diastereomers of the compound, even more preferably greater than about 95% free of other enantiomers or diastereoiners of the compound, even more highly preferably greater than about 98% free of other enantiomers or diastereomers of the compound and most preferably greater than about 99% flee of other enantiomers or diastereomers of the compound. Where the stereochemistry of the chiral centers present in the chemical structures illustrated herein is not specified, the chemical structure is intended to encompass compounds containing either stereoisomer of each chiral center present in the compound.

One example of some of the possible stereoisomers of the compounds of this invention is represented by formula (Ic):

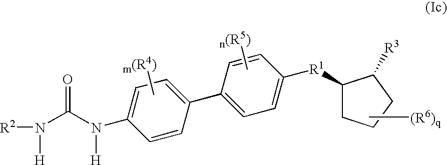

It is understood that embodiments of R¹, R², R³, R⁴, R⁵ and R⁶, and combinations of embodiments, including preferred, more preferred and most preferred embodiments as described in formula (I) are also contemplated for compounds of formula (Ic).

Therapeutic compositions of the present compounds include an effective amount of the same formulated with one or more pharmaceutically acceptable carriers. The term “pharmaceutically acceptable carriers” as used herein, represents a non-toxic, solid, semi-solid or liquid filler, diluent, encapsulating material, or formulation auxiliary of any type. Examples of therapeutically suitable excipients include sugars; cellulose and derivatives thereof; oils; glycols; solutions; buffering, coloring, releasing, coating, sweetening, flavoring, and perfuming agents; and the like. These pharmaceutical compositions can be administered parenterally, intracisternally, orally, rectally, or intraperitoneally.

Liquid dosage forms for oral administration of the present compounds include formulations of the same as emulsions, microemulsions, solutions, suspensions, syrups, and elixirs. In addition to the compounds, the liquid dosage forms can contain diluents and/or solubilizing or emulsifying agents. Besides inert diluents, the oral compositions can include wetting, emulsifying, sweetening, flavoring, and perfuming agents.

Injectable preparations of the present compounds include sterile, injectable, aqueous and oleaginous solutions, suspensions or emulsions, any of which can be optionally formulated with parenterally suitable diluents, dispersing, wetting, or suspending agents These injectable preparations can be sterilized by filtration through a bacterial-retaining filter or formulated with sterilizing agents that dissolve or disperse in the injectable media.

Inhibition of DGAT-1 by the compounds of the present invention can be delayed by using a liquid suspension of crystalline or amorphous material with poor water solubility. The rate of absorption of the compounds depends upon their rate of dissolution, which, in turn, depends on their crystallinity. Delayed absorption of a parenterally administered compound can be accomplished by dissolving or suspending the compound in oil Injectable depot forms of the compounds can also be prepared by microencapsulating the same in biodegradable polymers Depending upon the ratio of compound to polymer and the nature of the polymer employed, the rate of release can be controlled. Depot injectable formulations are also prepared by entrapping the compounds in liposomes or microemulsions that are compatible with body tissues.

Solid dosage forms for oral administration of the present compounds include capsules, tablets, pills, powders, and granules. In such forms, the compound is mixed with at least one inert, pharmaceutically acceptable carrier such as a filler, extender, disintegrating agent, solution-retarding agent, wetting agent, absorbent, or lubricant. With capsules, tablets, and pills, the pharmaceutically acceptable carriers can also contain buffering agents. Suppositories for rectal administration can be prepared by mixing the compounds with a suitable non-irritating excipient that is solid at ordinary temperature but fluid in the rectum.

The present compounds can be microencapsulated with one or more of the carriers discussed previously. The solid dosage forms of tablets, dragees, capsules, pills, and granules can be prepared with coatings and shells such as enteric and release controlling In these forms, the compounds can be mixed with at least one inert diluent and can optionally include tableting lubricants and aids. Capsules can also optionally contain opacifying agents that delay release of the compounds in a desired part of the intestinal tract.

Transdermal patches have the added advantage of providing controlled delivery of the present compounds to the body. Such dosage forms are prepared by dissolving or dispensing the compounds in the proper medium Absorption enhancers can also be used to increase the flux of the compounds across the skin, and the rate of absorption can be controlled by providing a rate controlling membrane or by dispersing the compounds in a polymer matrix or gel.

Disorders that can be treated or prevented in a patient by administering to the patient, a therapeutically effective amount of compound of the present invention in such an amount and for such time as is necessary to achieve the desired result. The term “therapeutically effective amount,” refers to a sufficient amount of a compound of formula (I) to effectively ameliorate disorders by inhibiting DGAT-1 at a reasonable benefit/risk ratio applicable to any medical treatment. The specific therapeutically effective dose level for any particular patient depends upon a variety of factors including the disorder being treated and the severity of the disorder; the activity of the compound employed; the specific composition employed; the age, body weight, general health, sex, and diet of the patient; the time of administration, route of administration, rate of excretion; the duration of the treatment; and drugs used in combination or coincidental therapy.

The total daily dose of the compounds of the present invention necessary to inhibit the action of DGAT-1 in single or divided doses can be in amounts, for example, from about 0.01 to 50 mg/kg body weight. In a more preferred range, compounds of the present invention inhibit the action of DGAT-1 in a single or divided doses from about 0.05 to 25 mg/kg body weight Single dose compositions can contain such amounts or submultiple doses thereof of the compounds of the present invention to make up the daily dose. In general, treatment regimens include administration to a patient in need of such treatment from about 1 mg to about 1000 mg of the compounds per day in single or multiple doses.

### Biological Data

Inhibition of DGAT-1

The identification of the compounds of the invention as DGAT-1 inhibitors was readily achieved using a high throughput screening FlashPlate assay In this assay, recombinant human DGAT-1 containing an N-terminal His₆-epitope tag was produced in the baculovirus expression system. Insect cells (e.g., Sf9 or High Five) were infected for 24 to 72 hr and collected by centrifugation. Cell pellets were resuspended in homogenization buffer [250 mM sucrose, 10 mM Tris-HCl (pH 7.4), 1 mM EDTA] and lysed using a homogenization apparatus, such as a Microfluidizer (single pass, 4° C.) Cell debris was removed by centrifugation at 10,000×g for 30 min, and microsonial membranes were collected by ultracentrifugation at 100,000×g for 30 min.

DGAT-1 activity was determined as followed: Assay buffer [20 mM HEPES (pH 7,5), 2 mM MgCl₂, 0.04% BSA] containing 50 μM of enzyme substrate (didecanoyl glycerol) and 7.5 μM radiolabeled acyl—CoA substrate.[1-¹⁴C]decanoyl—CoA) was added to each well of a phospholipid FlashPlate (PerkinElmer Life Sciences). A small aliquot of membrane (1 μg/well) was added to start the reaction, which was allowed to proceed for 60 min. The reaction was terminated upon the addition of an equal volume (100 μL) of isopropanol The plates were sealed, incubated overnight and counted the next morning on a TopCount Scintillation Plate Reader (PerkinElmer Life Science). DGAT-1 catalyzes the transfer of the radiolabeled decanoyl group onto the sn-3 position of didecanoyl glycerol. The resultant radiolabeled tridecanoyl glycerol (tricaprin) preferentially binds to the hydrophobic coating on the phospholipid FlashPlate. The proximity of the radiolabeled product to the solid scintillant incorporated into the bottom of the FlashPlate induced fluor release from the scintillant, which was measured in the TopCount Plate Reader. Various concentrations (e.g. 0.0001 μM, 0,001 μM, 0.01 μM, 0.1 μM, 1.0 μM, 10.0 μM) of the representative compounds of the invention were added to individual wells prior to the addition of membranes The potencies of DGAT-1 inhibition for the compounds of the present invention were determined by calculating the IC₅₀ values defined as the inhibitor concentration from the sigmoidal dose response curve at which the enzyme activity was inhibited 50%. Compounds of the present invention were effective in inhibiting DGAT-1 activity and thus are useful as therapeutic agents for treating conditions and diseases that are associated with DGAT-1 activity.

TABLE 1
DGAT-1 Inhibition of Compounds of
the Present Invention (IC₅₀ nM)

| Compound | IC₅₀ nM |
|---|---|
| A | 26 |
| B | 133 |
| C | 53 |
| D | 9 |
| E | 6.4 |
| F | 471 |
| G | 137 |
| H | 115 |
| I | 1160 |
| J | 474 |
| K | 20 |
| L | 69 |
| M | 10 |
| N | 13 |
| O | 55 |
| P | 20 |
| Q | 42 |
| R | 56 |
| S | 98 |
| T | 11 |
| U | 7.4 |
| V | 101 |
| W | 39 |
| X | 157 |
| Y | 34 |
| Z | 17 |
| AA | 7.6 |
| AB | 9.4 |
| AC | 384 |
| AD | 322 |
| AE | 3770 |
| AF | 15 |
| AG | 64 |
| AH | 16 |
| AI | 133 |
| AJ | 202 |
| AK | 93 |
| AL | 34 |
| AM | 10 |
| AN | 40 |
| AO | 40 |
| AP | 43 |
| AQ | 22 |
| AR | 88 |
| AS | 37 |
| AT | 272 |
| AU | 22 |
| AV | 10 |
| AW | 10 |
| AX | 42 |
| AY | 39 |
| AZ | 119 |
| BA | 200 |
| BB | 818 |
| BC | 33 |
| BD | 8 |
| BE | 63 |
| BF | 1738 |
| BG | 21 |
| BH | 88 |
| BI | 60 |
| BJ | 154 |
| BK | 17 |
| BL | 279 |
| BM | 218 |

Evaluation of Compound Efficacy on the Reduction of Body Weight in Diet-Induced Obese Mice

The purpose of this protocol was to determine the effect of chronic administration of a compound on body weight and other metabolic disease parameters in mice made obese by spontaneous ad libitum consumption of a high-fat diet. Diet-induced obesity (DIO) in rodents mimics key aspects of human obesity and metabolic syndrome DIO mice used in this study have been shown to be hyperinsulinemic and insulin resistant, hyperleptinemic and leptin resistant, and have marked visceral obesity (for review on DIO mice see Collins et al., Physiol. Behav. 81:243-248, 2004),

Individually housed male C57BL/6J mice were given ad lib access to water and to either a low fat diet (D12450B) or a high-fat content diet (D12492 containing 60% kcal from fat, both from Research Diets Inc., New Brunswick, N.J.), for approximately 18 weeks. Mice were sham dosed once daily with the study vehicle for 7 days prior to active dosing to acclimate them to handling and oral gavage. One day prior to active compound dosing, mice were assigned to groups of equal mean body weight and variance. A typical experiment consisted of 80-100 animals, 10 animals per dose including vehicle dosed low-fat and high-fat diet groups. Body weight and food intake were measured by differential weighing.

Representative compounds of the invention were typically dosed at 3, 10, or 30 mg/kg p.o. b.i.d. as a formulation in 1% Tween 80 in water, and the compounds were considered to be active if a statistically significant reduction in body weight was observed for the treated animals after a treatment period of at least seven days, relative to vehicle-treated control animals. In this mode, representative compounds produced a statistically significant reduction in body weight after a treatment period of at least seven days, relative to vehicle-treated control animals.

Liver triacylglycerides levels from DIO-mice treated with compounds of the invention typically dosed at 3, 10, or 30 mg/kg p.o, b.i.d. as a formulation in 1% Tween 80 in water for a treatment period of at least seven days were measured from ethanol extracted liver samples using Infinity™ reagents (Thermo Electron Corporation, Louisville. Colo., USA). Representative compounds of the invention produced a statistically significant reduction in liver triacylglycerides in DIO-mice after a treatment period of at least seven days, relative to vehicle treated control animals.

An insulin tolerance test was also performed at the end of study in DIO mice after a 4 hour fast. Blood glucose levels were monitored via tail snip before and at 30 minute intervals following a single i.p. injection of 0.25 U/kg insulin (Humulin-R, Lilly) using a Precision PCx glucose monitor (Abbott Laboratories. Abbott Park, Ill.). Representative compounds of the invention produced a statistically significant reduction in blood glucose in animals that had been treated for at least seven days, relative to vehicle-treated control animals.

The effect of co-dosing representative compounds of the invention with rimonabant was also evaluated in DIO-mice. Compounds of the invention were typically dosed at 3, 10, or 30 mg/kg p.o, b.i.d, as a formulation in 1% Tween 80 in water and rimonabant was typically co-administered at a dose of 3 or 10 mg/kg p.o. q.d as a formulation in 1% Tween in water. Compounds were considered to be active if they significantly decreased body weight compared to DIO-mice dosed with rimonabant alone. In this model, representative compounds produced a statistically significant reduction in body weight after a treatment period of at least seven days, relative to animals treated with rimonabant alone.

The effect of co-dosing representative compounds of the invention with fenofibrate was also evaluated in DIO-mice. Compounds of the invention were typically dosed at 3, 10, or 30 mg/kg p.o., b.i.d. as a formulation in 1% Tween 80 in water and fenofibrate was typically co-administered at a dose of 100 mg/kg p.o. b.i.d. as a formulation in 1% Tween in water. Compounds were considered to be active if they significantly decreased body weight compared to DIO-mice dosed with fenofibrate alone. In this model, representative compounds produced a statistically significant reduction in body weight after a treatment period of at least seven days, relative to animals treated with fenofibrate alone.

Accordingly, one aspect of the present invention provides a method of treating various conditions in a patient (such as mammal, preferably human) which includes administering to the patient a composition containing a compound of formula (I), (Ia), (Ib) or (Ic), or a pharmaceutically acceptable salt, prodrug, salt of a prodrug, or a combination thereof, in an amount that is effective in treating such conditions.

Another aspect of the present invention provides methods for treating obesity and inducing weight loss in an individual which includes administering to the individual a compound of the invention, or its pharmaceutically acceptable salt, prodrug, salt of a prodrug, or a combination thereof, in an amount that is effective in treating obesity or to induce weight loss. The invention further provides a method treating obesity and inducing weight loss in an individual which includes administering to the individual a pharmaceutical composition including a compound of the invention, or its pharmaceutically acceptable salt, prodrug, salt of a prodrug, or a combination thereof, in an amount that is effective in treating obesity or to induce weight loss, and a pharmaceutically acceptable carrier. Yet another aspect of the invention provides a method for preventing weight gain in an individual by administering at least one compound of the invention, or its pharmaceutically acceptable salt, prodrug, salt of a prodrug, or a combination thereof, in an amount that is sufficient to prevent weight gain.

The present invention also relates to the use of the compounds of this invention for the treatment of obesity-related diseases including associated dyslipidemia and other obesity- and overweight-related complications such as, for example, cholesterol gallstones, gallbladder disease, gout, cancer (erg., colon, rectum, prostate, breast, ovary, endometrium, cervix, gallbladder, and bile duct), menstrual abnormalities, infertility, polycystic ovaries, osteoarthritis, and sleep apnea, as well as for a number of other pharmaceutical uses associated therewith, such as the regulation of appetite and food intake, dyslipidemia, hypertriglyceridemia, Syndrome X, type 2 diabetes (non-insulin-dependent diabetes), atherosclerotic diseases such as heart failure, hyperlipidemia, hypercholesteremia, low HDL levels, hypertension, cardiovascular disease (including atherosclerosis, coronary heart disease, coronary artery disease, and hypertension), cerebrovascular disease such as stroke, and peripheral vessel disease. The compounds of this invention can also be useful for treating physiological disorders related to, for example, regulation of insulin sensitivity, inflammatory response, liver steatosis, elevated liver triacylglycerides, non-alcoholic fatty liver disease, non-alcoholic steatohepatitis, plasma triacylglycerides, HDL, LDL and cholesterol levels and the like.

Compounds of Formula (I), (Ia), (Ib) or (Ic) can be administered alone or in combination with one or more additional therapeutic agents Combination therapy includes administration of a single pharmaceutical dosage formulation which contains a compound of Formula (I), (Ia), (Ib) or (Ic) and one or more additional therapeutic agents, as well as administration of the compound of Formula (I), (Ia), (Ib) or (Ic), and each additional therapeutic agents in its own separate pharmaceutical dosage formulation. For example, a compound of Formula (I), (Ia), (Ib) or (Ic) and a therapeutic agent can be administered to the patient together, in a single oral dosage composition having a fixed ratio of each active ingredient, such as a tablet or capsule, or each agent can be administered in separate oral dosage formulations.

Where separate dosage formulations are used, the compound of Formula (I). Formula (I), (Ia), (Ib) or (Ic) and one or more additional therapeutic agents can be administered at essentially the same time (e.g., concurrently) or at separately staggered times (e.g., sequentially).

For example, the compounds of Formula (I), (Ia), (Ib) or (Ic) can be used in combination with other therapies and drugs useful for the treatment of obesity. For example, anti-obesity drugs include β-3 agonists such as CL-316,243; CB-1 antagonists (for example, rimonabant); neuropeptide Y5 inhibitors; appetite suppressants, such as, for example, sibutramine (Meridia®); and lipase inhibitors, such as, for example, orlistat (Xenical™). The compounds of the present invention can also be administered in combination with a drug compound that modulates digestion and/or metabolism such as drugs that modulate thermogenesis, lipolysis, gut motility, fat absorption, and satiety.

In addition, the compounds of Formula (I), (Ia), (Ib) or (Ic) can be administered in combination with one or more of the following agents for the treatment of diabetes or diabetes-related disorders including PPAR ligands (agonists, antagonists), insulin secretagogues, for example, sulfonylurea drugs and non-sulfonylurea secretagogues, α-glucosidase inhibitors, insulin sensitizers, hepatic glucose output lowering compounds, and insulin and insulin derivatives. Such therapies can be administered prior to, concurrently with, or following administration of the compounds of the invention. Insulin and insulin derivatives include both long and short acting forms and formulations of insulin. PPAR ligands can include agonists and/or antagonists of any of the PPAR receptors or combinations thereof. For example, PPAR ligands can include ligands of PPAR-α, PPAR-γ, PPAR-δ or any combination of two or three of the receptors of PPAR. PPAR ligands include, for example, rosiglitazone, troglitazone, and pioglitazone. Sulfonylurea drugs include, for example, glyburide, glimepiride, chlorpropamide, tolbutamide, and glipizide. α-glucosidase inhibitors that can be useful in treating diabetes when administered with a compound of the invention include acarbose, miglitol, and voglibose. Insulin sensitizers that can be useful in treating diabetes include PPAR-γ agonists such as the glitazones (e.g, troglitazone, pioglitazone, englitazone, MCC-555, rosiglitazone, and the like) and other thiazolidinedione and non-thiazolidinedione compounds; biguanides such as metformin and phenformin; protein tyrosine phosphatase-1B (PP-1B) inhibitors; dipeptidyl peptidase IV (DPP-IV) inhibitors, and 11beta-HSD inhibitors. Hepatic glucose output lowering compounds that can be useful in treating diabetes when administered with a compound of the invention include glucagon antagonists and metformin, such as Glucophage and Glucophage XR. Insulin secretagogues that can be useful in treating diabetes when administered with a compound of the invention include sulfonylurea and non-sulfonylurea drugs: GLP-1, GIP, PACAP, secretin, and derivatives thereof; nateglinide, meglitinide, repaglinide, glibenclamide, glimepiride, chlorpropamide, glipizide. GLP-1 includes derivatives of GLP-1 with longer half-lives than native GLP-1, such as, for example, fatty-acid derivatized GLP-1 and exendin.

Compounds of the invention can also be used in methods of the invention in combination with drugs commonly used to treat lipid disorders in patients. Such drugs include, but are not limited to, HMG-CoA reductase inhibitors, nicotinic acid, fatty acid lowering compounds (e.g., acipimox); lipid lowering drugs (e.g., stanol esters, sterol glycosides such as tiqueside, and azetidinones such as ezetimibe). ACAT inhibitors (such as avasimibe), bile acid sequestrants, bile acid reuptake inhibitors, microsomal triacylglycerides transport inhibitors, and fibric acid derivatives. HMG-CoA reductase inhibitors include, for example, lovastatin, simvastatin, pravastatin, fluvastatin, atorvastatin, rivastatin, itavastatin, cerivastatin, and ZD-4522. Fibric acid derivatives include, for example, clofibiate, fenofibtate, bezafibrate, ciprofibrate, beclofibrate, etofibrate, and gemfibrozil. Sequestrants include, for example, cholestyramine, colestipol, and dialkylaminoalkyl derivatives of a cross-linked dextran.

Compounds of the invention can also be used in combination with anti-hypertensive drugs, such as, for example, β-blockers and ACE inhibitors. Examples of additional anti-hypertensive agents for use in combination with the compounds of the present invention include calcium channel blockers (L-type and T-type; e.g., diltiazem, verapamil, nifedipine, amlodipine and mybefradil), diuretics (e.g., chlorothiazide, hydrochlorothiazide, flumethiazide, hydroflumethiazide, bendroflumethiazide, methylehlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors. ACE inhibitors (e.g., captopril, zofenopril, fosinopril, enalapril, ceranopril, cilazopril, delapril, pentopril, quinapril, ramipril, lisinopril). AT-1 receptor antagonists (e.g., losartan, irbesartan, valsartan), ET receptor antagonists (e.g., sitaxsentan, atrsentan, neutral endopeptidase (NEP) inhibitors, vasopepsidase inhibitors (dual NEP-ACE inhibitors) (e.g., omapatrilat and gemopatrilat), and nitrates.

The compounds of this invention can also be co-administered with an incretin mimetic such as, but not limited to, exenatide.

Synthetic Methods

The compounds and processes of the present invention will be better understood in connection with the following synthetic schemes, which together illustrate the methods by which the compounds of the invention can be prepared. The synthesis of compounds of formula (I) wherein the groups R¹, R², R³, R⁴, R⁵, R⁶, m, n, p, and q are as set forth in the summary of the invention unless otherwise noted, is exemplified in Schemes 1 and 2.

Optimum reaction conditions and reaction times for each individual step can vary depending on the particular reactants employed and substituents present in the reactants used. Unless otherwise specified, solvents, temperatures and other reaction conditions can be readily selected by one of ordinary skill in the art. Specific procedures are provided in the Synthetic Examples section. Reactions can be worked up in the convention manner, e g, by eliminating the solvent from the residue and further purified according to methodologies generally known in the art such as, but are not limited to, crystallization, distillation, extraction, trituration and chromatography. Unless otherwise described, the starting materials and reagents are either commercially available or can be prepared by one skilled in the art from commercially available materials using methods described in the chemical literature.

This invention is intended to encompass compounds having formula (I) when prepared by synthetic processes or by metabolic processes. Preparation of the compounds of the invention by metabolic processes include those occurring in the human or animal body (in vivo) or processes occurring in vitro.

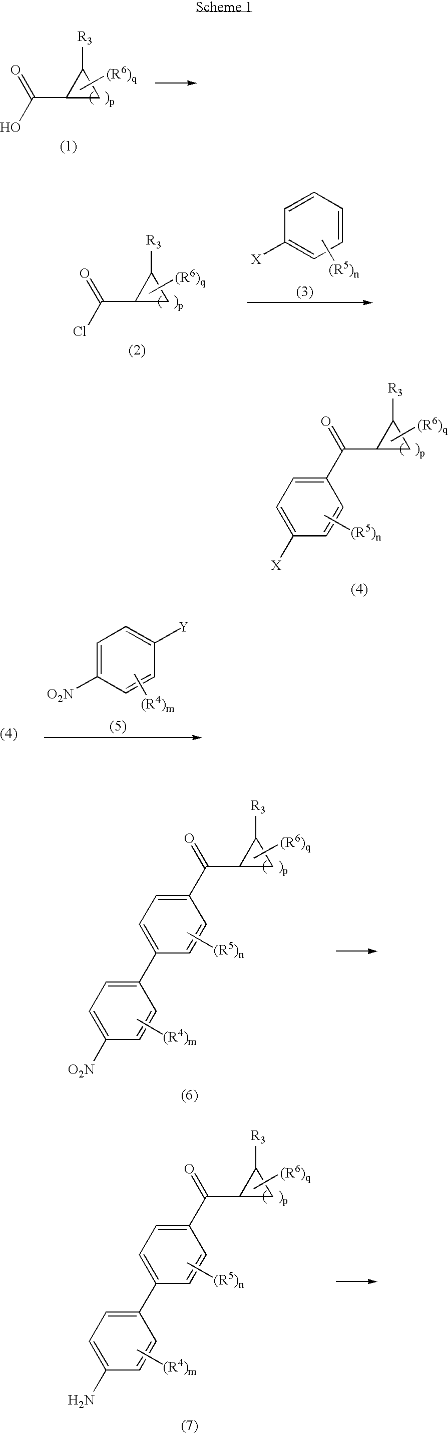

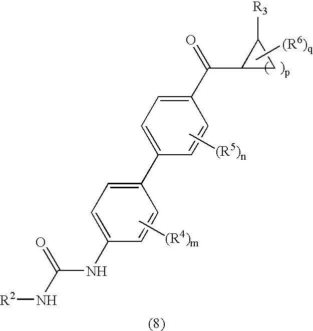

Acids of formula (1) can react with a chlorinating agent in a solvent such as, but not limited to, dichloromethane, at a temperature from about room temperature to about 50° C., to provide acid chlorides of formula (2). Non-limiting examples of the chlorinating agents include phosphorus pentachloride, oxalyl chloride and thionyl chloride/catalytic N,N-dimethylformamide. The acid chloride can be treated with an aryl of formula (3) wherein X is halogen or triflate in the presence of aluminum chloride, in a solvent such as, but not limited to, dichloromethane or dichloroethane to provide a compound of formula (4). In some instances, the aryl of formula (3) can act as the reactant as well as the reaction solvent. The reaction is generally conducted at a temperature ranging from about 0° C. to about 10° C.

Compounds of formula (4) can be treated with botonic acids or esters of formula (5) wherein Y is —B(OR₁₀₁)₂ and R₁₀₁ is hydrogen or C₁₋₆ alkyl, in the presence of a palladium catalyst, in a solvent such as, but not limited to, toluene, dioxane, N,N-dimethylformamide, N,N-dimethyl acetamide, dimethoxyethane, isopropanol, ethanol, water, or mixture thereof, to provide compounds of formula (6). Non-limiting examples of palladium catalyst suitable for the transformation include, tetrakis(triphenylphosphine)palladium(0) and bis(triphenylphosphine)palladium (II) chloride. The reaction can be facilitated with the addition of a base such as, but not limited to, potassium iodide, triethylamine, cesium carbonate, or sodium carbonate. The reaction is generally conducted at an elevated temperature such as 50° C. to about 100° C. and optionally in a microwave oven.

Compounds of formula (4) can also be treated with stannanes of formula (5) wherein X is —Sn(C₁₋₆ alkyl)₃, in the presence of a palladium catalyst such as, but not limited to, tetrakis(triphenylphosphine)palladium (0), and cesium fluoride, at elevated temperature, in a solvent such as dioxane. The transformations can also be effected by heating in a microwave reactor.

Conversely, compounds of formula (4) wherein X is B(OR¹⁰¹)₂ or Sn(C₁₋₆ alkyl)₃, can be treated with compounds of formula (5) wherein Y is halogen or triflate, using reaction conditions as set forth in the preceding paragraphs to provide compounds of formula (6). While many stannanes and boronic acidsor esters are commercially available, compounds of formula (4) and (5) wherein X and Y are independently B(OR₁₀₁)₂ or Sn(C₁₋₆ alkyl)₃ can also be prepared from reaction of compounds of formula (4) or (5) wherein X and Y are independently halogen or triflate, with boronic acids or distannanes of formula ((C₁₋₆ alkyl)₃Sn)₂, in the presence of a palladium catalyst, using methodologies that are known in the art.

Compounds of formula (6) can be treated with a reducing agent in a suitable solvent such as ethanol and water, at elevated temperature (generally at about 70° C. to about 100° C.) to provide anilines of formula (7). Examples of reducing agent include, but not limited to, iron powder and ammonium chloride, or hydrogen gas and palladium catalyst (egg 5-10% palladium on carbon, and 20% palladium hydroxide on carbon). Reaction of compounds of formula (7) with isocyanates of formula R²NCO, in a solvent such as, but not limited to, tetrahydrofuran, at about room temperature, provides compounds of formula (8).

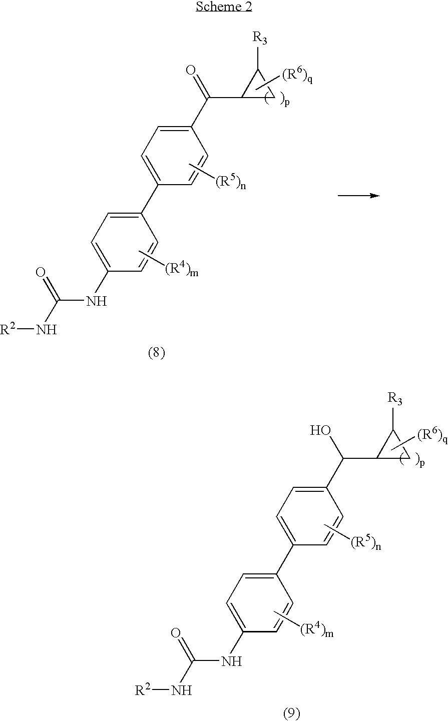

Compounds of formula (9) can be treating compounds of formula (8) with a reducing agent such as, but not limited to, sodium borohydride, in a suitable solvent such as, but not limited to, methanol, at about room temperature.

The present invention will now be described in connection with certain preferred embodiments which are not intended to limit its scope On the contrary, the present invention covers all alternatives, modifications, and equivalents as can be included within the scope of the claims. Routine experimentation, including appropriate manipulation of the reaction conditions, reagents used and sequence of the synthetic route, protection of any chemical functionality that can not be compatible with the reaction conditions, and deprotection at suitable point in the reaction sequence of the method are included in the scope of the invention. Suitable protecting groups and the methods for protecting and deprotecting different substituents using such suitable protecting groups are well know to those skilled in the art; examples of which can be found in T. Greene and P. Wuts, Protecting Groups in Chemical Synthesis (3^{rd} ed.), John Wiley & Sons, NY (1999), which is incorporated herein by reference in its entirety Synthesis of the compounds of formula (I) can be accomplished by methods analogous to those described above and in the following examples The following examples, which include preferred embodiments, will illustrate the preferred practice of the present invention, it being understood that the examples are for the purpose of illustration of certain preferred embodiments and are presented to provide what is believed to be the most useful and readily understood description of its procedures and conceptual aspects Finally, the compounds of the invention were named by ACD/ChemSketch version 5.06 (developed by Advanced Chemistry Development, Inc. Toronto, ON, Canada) or were given names consistent with ACD nomenclature.

### EXAMPLES

### Example 1

### methyl (1R,2R)-2-({4′-[({[4-(trifluoromethoxy)phenyl]amino}carbonyl)amino]-1,1′-biphenyl-4-yl}carbonyl)cyclopentanecarboxylate

### Example 1A

### cis-cyclopentane-1,2-dicarboxylic acid

To a solution containing ethyl 2-oxocyclohexanecarboxylate (100 g, 0.588 mol) in 300 mL of chloroform at 0° C. bromine (94 g, 0.588 mol) was added. The mixture was stirred overnight, washed with a saturated sodium bicarbonate solution and brine, dried over anhydrous sodium sulfate, filtered and concentrated under reduced pressure to remove the solvent. The residue was added drop wise to an ice-cold solution containing potassium hydroxide (140 g, 2.50 mol) in 800 mL of water, the reaction mixture was stirred for 2 hours and was extracted with diethyl ether. The aqueous phase was acidified with a 4.0 M hydrochloric acid solution and extracted with diethyl ether. The combined ethereal solution was dried over anhydrous sodium sulfate, filtered and evaporated under reduced pressure to give a yellow oil, which crystallized on standing to provide to Example 1A as colorless crystals.

### Example 1B

### cyclopentane-1,2-dicarboxylic anhydride

A solution containing Example 1A (56.6 g, 0.358 mol) in 1500 mL of acetic anhydride was heated at reflux for 20 hours. The excess acetic anhydride was removed by distillation under reduced pressure. The oily residue was distilled to give Example 1B as a colorless oil. ¹H NMR (500 MHz, DMSO-d₆) δ ppm 1.07-2.02 (m, 6H) and 3.47-3 55 (m, 2H).

### Example 1C

### cis-2-(methoxycarbonyl)cyclopentanecarboxylic acid

Example 1B (40.2 g, 286.9 mmol) was dissolved in methanol (250 mL), and the mixture was then heated at 50-55° C. under N₂ for 16 hours. Methanol was removed by rotary evaporation, and the residue was dried in vacuo to afford the desired product as a colorless oil

¹H NMR (500 MHz, DMSO-d6) δ ppm 1.54-1.61 (m, 1H), 1.66-1.76 (m, 1H), 1.80-1.91 (m, 4H), 3.95-3.02 (m, 2H), 3.54 (s, 3H), 12.05 (hr s, 1H)

### Example 1D

### methyl cis-2-(4-bromobenzoyl)cyclopentanecarboxylate

Step A:

A solution of Example 1C (30.7 g, 178.3 mmol), SOC₂ (39 mL, 535 mmol), and N,N-dimethylformamide (0.33 mL) in 383 mL CH₂Cl₂ was stirred at room temperature overnight under N₂. The solvent was removed by rotary evaporation at <40° C., and the residue was dried in vacuo for 1 hour.

Step B:

The intermediate described in step A was dissolved in bromobenzene (112.5 mL, 1.067 mol), and AlCl₃ (47.5 g, 357 mmol) was then added portion wise at <5° C. The reaction mixture turned dark brown, and was stirred at <5° C. for 4 hours under N₂. NMR showed that little starting material remained. The reaction mixture was then slowly poured into 700 mL ice-water, and then 350 mL ethyl acetate was added. After the mixture was stirred for 10 minutes, the aqueous (top) layer was separated, and extracted with 200 mL ethyl acetate. The combined organic layers were washed with water (2×350 mL) and saturated NaHCO₃ solution (70 mL), dried over Na₂SO₄, and filtered. Removal of solvent and drying in vacuo provided the desired product, which was used directly in the next step. ¹H NMR (500 MHz, DMSO-d₆) δ ppm 1.58-1.83 (m, 3H), 1.91-2.05 (m, 3H), 3.15-3.21 (m, 1H), 3.37 (s, 3H), 4.13-4.19 (m, 1H), 7.73 (d, J=8.59 Hz, 2H), 7.87 (d, J=8.90 Hz, 2H).

### Example 1E

### trans-2-(4-bromobenzoyl)cyclopentanecarboxylic acid

A solution of Noah (42.9g, 1.07 mol) in 234 mL water was added to a solution of Example 1D (178.3 mmol) in methanol (234 mL). The reaction mixture was stirred at room temperature overnight. NMR showed that little starting material remained. After 350 mL, solvent was removed by rotary evaporation, the mixture was diluted with 350 mL water. Conc. Ha1 was slowly added with stirred at <15° C., to adjust the acidity to pH <6. A precipitate formed, and stirring was continued for 1 hour. The solid precipitate was filtered, and rinsed with water. The dried filter cake was dissolved in 350 mL ethyl acetate, dried over Na₂SO₄, and filtered. Removal of solvent and drying in vacuo afforded the title compound. ¹H NMR (500 MHz, DMSO-d₆) δ ppm 1.55-1.85 (m, 4H), 1.93-2.03 (m, 1H), 2.09-2.15 (m, 1H), 3.15-3.21 (m, 1H), 4.00-4.06 (m, 1H), 7.75 (d, J=8.59 Hz, 2H), 7.93 (d, J=8.59 Hz, 2H), 12.21 (br s, 1H).

### Example 1F

### (1R,2R)-2-(4-bromobenzoyl)cyclopentanecarboxylic acid

A mixture of Example 1E (27.4 g, 92.2 mmol) and (R)-(+)-alpha-methyl-benzylamine (5.59 g, 46.1 mmol) in CH₃CN (268 mL) was heated at 90-95° C. under N₂ to provide a solution. The hot solution was allowed to cool slowly with slow stirring overnight. The crystallized solid was filtered, and rinsed with CH₃CN (12 mL). Drying in vacuo to a constant weight. The solid was then dissolved in a mixture of solvent (62 mL ethanol and 124 mL water) with heating at 90-95° C. under N₂. The hot solution was allowed to cool slowly with slow stirring overnight. The solid formed was filtered, and rinsed with 15 mL of 1:2 ethanol/water. After drying in vacuo to a constant weight, this white solid was stirred with 1N HCl (120 mL) and ethyl acetate (120 mL) for 10 minutes. The organic layer was separated, washed with water (2×48 mL), and dried over Na₂SO₄. Removal of solvent and drying in vacuo provided an off-white solid (>94% EE based on chiral HPLC). Chiral HPLC method: Chiracel OJ analytical column, 2:98 ethanol/hexanes (both containing 0.1% trifluoroacetic acid), 1.5 mL/min flow rate, retention times were 18.90 min and 22.18 minutes for the (S,S) and (R,R) isomers, respectively. ¹H NMR (500 MHz, DMSO-d₆) δ ppm 1.55-1.85 (m, 4H), 1.93-2.03 (m, 1H), 2.09-2.15 (m, 1H), 3.15-3.21 (m, 1H), 4.00-4.06 (m, 1H), 7.75 (d, J=8.59 Hz, 2H), 7.93 (d, J=8.59 Hz, 2H), 12.21 (br s, 1H).

### Example 1G

### methyl (1R,2R)-2-(4-bromobenzoyl)cyclopentanecarboxylate

A suspension of Example 1F (7.96 g, 26.8 mmol), iodomethane (2.5 mL, 40.2 mmol), and NaHCO₃ (6.75g, 80.4 mmol) in 94 mL N,N-dimethylformamide was stirred at Room temperature under N₂ overnight. NMR showed that little starting material remaining. Water (262 mL) was added to the reaction mixture, and then concentrated. HCl was slowly added with stirring at <15° C. to adjust the acidity to pH <7. Ethyl acetate (300 mL) was added to extract the product out. The aqueous layer was extracted with ethyl acetate (2×200 mL). The combined organic layers were washed with brine, dried over Na₂SO₄ and concentrated. The residue was purified on a flash column to, eluting with 0-5% ethyl acetate in hexanes, to provide the title compound. ¹H NMR (500 MHz, DMSO-d₆) δ ppm 1.59-1.84 (m, 4H), 1.97-2.04 (m, 1H), 2.11-2.19 (m, 1H), 3.24-3.29 (m, 1H), 3.56 (s, 3H), 4.02-4.08 (m, 1H), 7.76 (d, J=8.90 Hz, 2H), 7.93 (d, J=8.59 Hz, 2H).

### Example 1H

### methyl (1R,2R)-2-[(4′-nitro-1,1′-biphenyl-4-yl)carbonyl]cyclopentanecarboxylate

To an ambient slurry of Example 1G (2 g, 6.4 mmol), 4-nitrophenyl boronic acid (2.1 g, 12.8 mmol) and KF (1.12 g, 19.3 mmol) in dimethoxyethane/toluene/ethanol/H₂O (10/1/6/3 ratio, 30 mL) was added to palladium tetrakis(triphenylphosphine) (75 mg, 0.06 mmol) in a single portion. The reaction was heated at 90° C. overnight, cooled to room temperature, filtered through celite, washed with ethyl acetate, concentrated and purified on a flash column, eluting with 0-15% ethyl acetate in hexanes to provide the title compound. ¹H NMR (500 MHz, DMSO-d₆) δ ppm 1.58-1.89 (m, 4H), 1.99-2.07 (m, 1H), 2.16-2.23 (m, 1H), 3.24-3.29 (m, 1H), 3.58 (s, 3H), 4.11-4.18 (m, 1H), 7.96 (d, J=8.59 Hz, 2H), 8.06 (d, J=8.90 Hz, 2H), 8.14 (d, J=8.59 Hz, 2H), 8.34 (d, J=8.90 Hz, 2H); MS (ESI) m/z 354.0 [M+H]⁺.

### Example 1I

### methyl (1R,2R)-2-[(4′-amino-1,1′-biphenyl-4-yl)carbonyl]cyclopentanecarboxylate

A mixture of Example 1H (2.206 g, 6.24 mmol), iron powder (1.046 g, 18.7 mmol), and NH₄Cl (334 mg, 6.24 mmol) in a mixture of solvents (90 mL of ethanol and 25 mL of water) was heated at 85° C. under N₂ for 2 hours. The reaction mixture was filtered through celite, treated with aqueous saturated sodium bicarbonate (50 mL) and extracted with ethyl acetate. The organic layer was washed with brine, dried over Na₂SO₄, filtered and concentrated. The residue was concentrated to provide the title compound without further purification. ¹H NMR (500 MHz, DMSO-d₆) δ ppm 1.54-1.86 (m, 4H), 1.98-2.07 (m, 1H), 2.13-2.22 (m, 1H), 3.25-3.29 (m, 1H), 3.57 (s, 3H), 4.04-4.10 (m, 1H), 5.41 (s, 2H), 6.86 (d, J=8.90 Hz, 2H), 7.48 (d, J=8.59 Hz, 2H), 7.70 (d, J=8.90 Hz, 2H), 7.98 (d, J=8.60 Hz, 2H); MS (ESI) m/z .324.0 [M+H]⁺.

### Example 1J

### methyl (1R, 2R)-2-({4′-[({[4-(trifluoromethoxy)phenyl]amino}carbonyl)amino]-1,1′-biphenyl-4-yl}carbonyl)cyclopentanecarboxylate

A scintillation vial was charged with Example 1I (50 mg, 0.155 mmol), 4-(trifluoromethoxy)phenyl isocyanate (31.4 mg, 0.155 mmol) and tetrahydrofuran (8 mL) and place in a shaker overnight at room temperature. The mixture was concentrated and purified on RP-HPLC (Preparative reversed-phase chromatography was performed using a Zorbax SB-C18 7 uM 21.2×250 mm column with UV detection analyzed at 220 and 254 nM. Preparative method: (water with 0.1% trifluoroacetic acid and CH₃CN with 0.1% trifluoroacetic acid gradient) 5-95% CH3CN over 30 minutes at 15 mL/min.) to provide the title product. ¹H NMR (500 MHz, DMSO-d₆) δ ppm 1.58-1.86 (m, 4H), 2.00-2.07 (m, 1H), 2.16-2.22 (m, 1H), 3.30-3.35 (m, 1H), 3.58 (s, 3H), 4.09-4.14 (m, 1H), 7.30 (d, J=8.24 Hz, 2H), 7.56-7.61 (m, 4H), 7.73 (d, J=8.54 Hz, 2H), 7.82 (d, J=8.55, 2H), 8.06 (d, J=8.54 Hz, 2H), 8.94 (d, J=9.46 Hz, 2H), MS (ESI) m/z 513.2 [M+H]⁺.

### Example 2

### (1R,2R)-2-({4′-[({[4-(trifluoromethoxy)phenyl]amino}carbonyl)amino]-1,1′-biphenyl-4-yl}carbonyl)cyclopentanecarboxylic acid

A scintillation vial was charged with Example 1J (53 mg, 0.1 mmol), LiOH (21 mg, 0.5 mmol) and 10 mL of 4:1 tetrahydrofuran/water and placed in a shaker overnight at room temperature. The mixture was concentrated and purified by RP-HPLC (Preparative reversed-phase chromatography was performed using a Zorbax SB-C18 7 uM 21.2×250 mm column with UV detection analyzed at 220 and 254 nM. Preparative method: (water with 0 1% trifluoroacetic acid and CH₃CN with 0.1% trifluoroacetic acid gradient) 5-95% CH3CN over 30 minutes at 15 mL/min.) to provide the title product. ¹H NMR (500 MHz, DMSO-d₆) δ ppm 1.59-1.86 (m, 4H), 1.99-2.05 (m, 1H), 2.14-2.21 (m, 1H), 3.20-3.25 (m, 1H), 4.07-4.15 (m, 1H), 7.30 (d, J=8.54 Hz, 2H), 7.56-7.61 (m, 4H), 7.72 (d, J=8.85 Hz, 2H), 7.82 (d, J=8.23 Hz, 2H), 8.07 (d, J=8.54 Hz, 2N), 8.94 (d, J=10.37 Hz, 2H), 12.23(s, 1H); MS (ESI) m/z 527.2 [M+H]⁺.

### Example 3

### methyl (1R,2R)-2-({4′-[({[4-(trifluoromethyl)phenyl]amino}carbonyl)amino]-1,1′-biphenyl-4-yl}carbonyl)cyclopentanecarboxylate

Example 3 was prepared using the procedure as described for Example 1J substituting 4-(trifluoromethyl)phenyl isocyanate for 4-(trifluoromethoxy)phenyl isocyanate. ¹H NMR (500 MHz, DMSO-d₆) δ ppm 1.58-1.86 (m, 4H), 2.00-2.07 (m, 1H), 2.17-2.23 (m, 1H), 3.30-3.35 (m, 1H), 3.58 (s, 3H), 4.09-4.14 (m, 1H), 7.61-7.69 (m, 6H), 7.74 (d, J=8.54 Hz, 2H), 7.83 (d, J=8.54 Hz, 2H), 8.06 (d, J=8.54 Hz, 2H), 9.01 (s, 1H), 9.16 (s, 1H); MS (ESI) m/z 511.2 [M+H]⁺.

### Example 4

### (1R,2R)-2-({4′-[({[4-(trifluoromethyl)phenyl]amino}carbonyl)amino]-1,140 -biphenyl-4-yl}carbonyl)cyclopentanecarboxylic acid

Example 4 was prepared using the procedure as described for Example 2 substituting Example 3 for Example 1J. ¹H NMR (500 MHz, DMSO-d₆) δ ppm 1.56-1.87 (m, 4H), 1.97-2.06 (m, 1H), 2.13-2.20 (m, 1H), 3.19-3.26 (m, 1H), 4.06-4.12 (m, 1H), 7.60-7.70 (m, 6H), 7.74 (d, J=8.60 Hz, 2H), 7.82 (d, J=8.29 Hz, 2H), 8.06 (d, J=8.59 Hz, 2H), 8.98 (s, 1H), 9.14 (s, 1H), 12.20 (s, 1H); MS (ESI) m/z 497.2 [M+H]⁺.

### Example 5

### methyl (1R,2R)-2-[(4′-{[(1,3-benzodioxol-5-ylamino)carbonyl]amino}-1,1′-biphenyl-4-yl)carbonyl]cyclopentanecarboxylate

Example 5 was prepared using the procedure as described for Example 1J substituting 3,4-(methylenedioxy)phenyl isocyanate for 4-(trifluoromethoxy)phenyl isocyanate. ¹H NMR (500 MHz, DMSO-d₆) δ ppm 1.57-1.88 (m, 4H), 1.99-2.06 (m, 1H), 2.15-2.23 (m, 1H), 3.25-3.35 (m, 1H), 3.58 (s, 3H), 4.08-4.14 (m, 1H), 5.97 (s, 2H), 6.79 (dd, J=8.29 Hz, 2.15 Hz, 1H), 6.84 (d, J=8.28 Hz, 1H), 7.21 (d, J=2.15 Hz, 1H), 7.58 (d, J=8.59 Hz, 2H), 7.70 (d, J=8.90 Hz, 2H), 7.81 (d, J=8.59 Hz, 2H), 8.05 (d, J=8.59 Hz, 2H), 8.59 (s, 1H), 8.78 (s, 1H), MS (ESI) m/z 487.2 [M+H]⁺.

### Example 6

### (1R,2R)-2-[4′-{[3-benzodioxol-5-ylamino)carbonyl]amino}-1,1′-biphenyl-4-yl)carbonyl]cyclopentanecarboxylic acid

Example 6 was prepared using the procedure as described for Example 2 substituting Example 5 for Example 1J. ¹H NMR (500 MHz, DMSO-d₆) δ ppm 1.59-1.86 (m, 4H), 1.98-2.05 (m, 1H), 2.14-2.21 (m, 1H), 3.20-3.25 (m, 1H), 4.06-4.11 (m, 1H), 5.98 (s, 2H), 6.78 (dd, J=6.41 Hz, 2.13 Hz, 1H), 6.84 (d, J=8.54 Hz, 1H), 7.22 (d, J=1.83 Hz, 1H), 7.58 (d, J=8.85 Hz, 2H), 7.71 (d, J=8.85 Hz, 2H), 7.81 (d, J=8.55 Hz, 2H), 8.06 (d, J=8.55 Hz, 2H), 8.64 (s, 1H), 8.82 (s, 1H); MS (ESI) m/z 473.2 [M+H]⁺.

### Example 7

### methyl (1R,2R)-2-{[4′-({[(4-acetylphenyl)amino]carbonyl}amino)-1,1′-biphenyl-4-yl]carbonyl}cyclopentanecarboxylate

Example 7 was prepared using the procedure as described for Example 1J substituting 4-acetylphenyl isocyanate for 4-(trifluoromethoxy)phenyl isocyanate. ¹H NMR (500 MHz, DMSO-d₆) δ ppm 1.57-1.89 (m, 4H), 2.00-2.08 (m, 1H), 2.16-2.24 (m, 1H), 3.28-3.35 (m, 1H), 3.58 (s, 3H), 4.08-4.14 (m, 1H), 7.59-7.62 (m, 4H), 7.73 (d, J=8.59 Hz, 2H), 7.83 (d, J=8.59 Hz, 2H), 7.92 (d, J=8.90 Hz, 2H), 8.06 (d, J=8.59 Hz, 2H), 8.99 (s, 1H), 9.15 (s, 1H); MS (ESI) m/z 485.2 [M+H]⁺.

### Example 8

### (1R,2R)-2-{[4′-({[(4-acetylphenyl)amino]carbonyl}amino)-1,1′-biphenyl-4-yl]carbonyl}cyclopentanecarboxylic acid

Example 8 was prepared using the procedure as described for Example 2 substituting Example 7 for Example 1J. ¹H NMR (500 MHz, DMSO-d₆) δ ppm 1.59-1.85 (m, 4H), 1.98-2.07 (m, 1H), 2.14-2.20 (m, 1H), 3.20-3.25 (m, 1H), 4.07-4.12 (m, 1H), 7.60-7.62 (m, 4H), 7.73 (d, J=8.55 Hz, 2H), 7.82 (d, J=8.55 Hz, 2H), 7.92 (d, J=8.85 Hz, 2H), 8.07 (d, J=8.84 Hz, 2H), 9.01 (s, 1H), 9.17 (s, 1H), 12.23 (s, 1H); MS (ESI) m/z 471.2 [M+H]⁺.

### Example 9

### methyl (1R,2R)-2-({4′-[(anilinocarbonyl)amino]-1,1′-biphenyl-4-yl}carbonyl)cyclopentanecarboxylate

A 50 ml round bottom flask was charged with Example 11 (900 mg, 2.78 mmol), phenyl isocyanate (365 mg, 3.06 mmol) and tetrahydrofuran (20 mL). The reaction was stirred overnight at room temperature. The reaction mixture was concentrated and purified by a flash column (5%-50% ethyl acetate in hexane) to provide the title product. ¹H NMR (500 MHz, DMSO-d₆) δ ppm 1.56-1.86 (m, 4H), 2.00-2.07 (m, 1H), 2.16-2.22 (m, 1H), 3.30-3.35 (m, 1H), 3.58 (s, 3H), 4.08-4.14 (m, 1H), 6.98 (t, J=7.37 Hz, 1H), 7.29 (t, J=7.67 Hz, 2H), 7.47 (d, J=7.67 Hz, 2H), 7.60 (d, J=8.90 Hz, 2H), 7.72 (d, J=8.90 Hz, 2H), 7.82 (d, J=8.59 Hz, 2H), 8.05 (d, J=8.59 Hz, 2H), 8.71 (s, 1H), 8.85 (s, 1H); MS (ESI) m/z 443.2 [M+H]⁺.

### Example 10

### (1R,2R)-2-({4′-[(anilinocarbonyl)amino]-1,1′-biphenyl-4-yl}carbonyl)cyclopentanecarboxylic acid

A 100 ml round bottom flask was charged with Example 9 (800 mg, 1.81 mmol), LiOH (380 mg, 9 mmol), and 50 mL of 4:1 tetrahydrofuran/water. The reaction was stirred overnight at room temperature, and then quenched by addition of 4 M HCl, adjusting the pH of the reaction mixture to <7. The mixture was concentrated and purified on RP-HPLC (Preparative reversed-phase chromatography was performed using a Zorbax SB-C18 7 uM 21.2×250 mm column with UV detection analyzed at 220 and 254 nM. Preparative method: (water with 0.1% trifluoroacetic acid and CH₃CN with 0.1% trifluoroacetic acid gradient) 5-95% CH3CN over 30 minutes at 15 mL/min.) to provide the title product. ¹H NMR (500 MHz, DMSO-d₆) δ ppm 1.58-1.86 (m, 4H), 1.98-2.05 (m, 1H), 2.14-2.21 (m, 1H), 3.20-3.24 (m, 1H), 4.07-4.12 (m, 1H), 6.99 (t, J=7.33 Hz, 1H), 7.29 (t, J=8.24 Hz, 2H), 7.48 (d, J=7.63 Hz, 2H), 7.60 (d, J=8.55 Hz, 2H), 7.72 (d, J=8.55 Hz, 2H), 7.82 (d, J=8.23 Hz, 2H), 8.06 (d, J=8.54 Hz, 2H), 8.73 (s, 1H), 8.87 (s, 1H), 12.23 (s, 1H); MS (ESI) m/z 429.1 [M+H]⁺.

### Example 11

### methyl (1R,2R)-2-{[4′-({[(2-chlorophenyl)amino]carbonyl}amino)-1,1′-biphenyl-4-yl}carbonyl}cyclopentanecarboxylate

Example 11 was prepared using the procedure as described for Example 1J, substituting 2-chlorophenyl isocyanate for 4-(trifluoromethoxy)phenyl isocyanate. ¹H NMR (500 MHz, DMSO-d₆) δ ppm 1.57-1.87 (m, 4H), 2.00-2.07 (m, 1H), 2.17-2.23 (m, 1H), 3.30-3.35 (m, 1H), 3.58 (s, 3H), 4.09-4.14 (m, 1H), 7.05 (t, J=7.63 Hz, 1H), 7.32 (t, J=7.01 Hz, 1H), 7.47 (dd, J=7.93 Hz, 1.53 Hz, 1H), 7.61 (d, J=8.85 Hz, 2H), 7.74 (d, J=8.55 Hz, 2H), 7.83 (d, J=8.54 Hz, 2H), 8.06 (d, J=8.54 Hz, 2H), 8.19 (d, J=8.24 Hz, 1H), 8.37 (s, 1H), 9.60 (s, 1H); MS (ESI) m/z 477.2 [M+H]⁺.

### Example 12

### (1R,2R)-2-{[4′-({[(2-chlorophenyl)amino]carbonyl}amino)-1,1′-biphenyl-4-yl]carbonyl}cyclopentanecarboxylic acid

Example 12 was prepared using the procedure as described for Example 2 substituting Example 11 for Example 1J. ¹H NMR (500 MHz, DMSO-d₆) δ ppm 1.63-1.88 (m, 4H), 1.92-1.99 (m, 1H), 2.07-2.11 (m, 1H), 3.07-3.12 (m, 1H), 4.13-4.18 (m, 1H), 7.05 (t, J=7.93 Hz, 1H), 7.31 (t, J=7.32 Hz, 1H), 7.45 (dd, J=6.72 Hz, 1.52 Hz, 1H), 7.64 (d, J=8.54 Hz, 2H), 7.71 (d, J=8.85 Hz, 2H), 7.81 (d, J=8.54 Hz, 2H), 8.08-8.12 (m, 3H), 8.92 (s, 1H), 10.21 (s, 1H); MS (ESI) m/z 463.1 [M+H]⁺.

### Example 13

### methyl (1R,2R)-2-{[4′-({[(3-chlorophenyl)amino]carbonyl}amino-1,1′-biphenyl-4-yl]carbonyl}cyclopentanecarboxylate

Example 13 was prepared using the procedure as described for Example 1J, substituting 3-chlorophenyl isocyanate for 4-(trifluoromethoxy)phenyl isocyanate. ¹H NMR (500 MHz, DMSO-d₆) δ ppm 1.56-1.87 (m, 4H), 2.00-2.07 (m, 1H), 2.16-2.23 (m, 1H), 3.30-3.34 (m, 1H), 3.58 (s, 3H), 4.09-4.14 (m, 1H), 7.02-7.05 (m, 1H), 7.28-7.33 (m, 2H), 7.60 (d, J=8.84 Hz, 2H), 7.72-7.74 (m, 3H), 7.82 (d, J=8.55 Hz, 2H), 8.06 (d, J=8.24 Hz, 2H), 8.96 (d, J=2.44, 2H); MS (EST) m/z 477.1 [M+H]⁺.

### Example 14

### (1R,2R)-2-{[4′-({[(3-chlorophenyl)amino]carbonyl}amino)-1,1′-biphenyl-4-yl]carbonyl}cyclopentanecarboxylic acid

Example 14 was prepared using the procedure as described for Example 2 substituting Example 13 for Example 1. ¹H NMR (500 MHz, DMSO-d₆) δ ppm 1.59-1.86 (m, 4H), 1.98-2.05 (m, 1H), 2.14-2.20 (m, 1H), 3.20-3.25 (m, 1H), 4.07-4.12 (m, 1H), 7.02-7.04 (m, 1H), 7.29-7.31 (m, 2H), 7.61 (d, J=8.85 Hz, 2H), 7.72-7.73 (m, 3H), 7.82 (d, J=8.54 Hz, 2H), 8.06 (d, J=8.23 Hz, 2H), 9.15 (s, 2H), 12.24 (s, 1H); MS (ESI) m/z 463.1 [M+H]⁺.

### Example 15

### methyl (1R,2R)-2-{[4′-({[(4-chlorophenyl)amino]carbonyl}amino)-1,1′-biphenyl-4-yl}carbonyl}cyclopentanecarboxylate

Example 15 was prepared using the procedure as described for Example 1J, substituting 4-chlorophenyl isocyanate for 4-(trifluoromethoxy)phenyl isocyanate. ¹H NMR (500 MHz, DMSO-d₆) δ ppm 1.56-1.87 (m, 4H), 1.99-2.07 (m, 1H), 2.16-2.23 (m, 1H), 3.30-3.35 (m, 1H), 3,58 (s, 3H), 4.09-4.14 (m, 1H), 7.34 (d, J=8.85 Hz, 2H), 7.51 (d, J=8.84 Hz, 2H), 7.60 (d, J=8.54 Hz, 2H), 7.72 (d, J=8.85 Hz, 2H), 7.82 (d, J=8.54 Hz, 2H), 8.06 (d, J=8.24 Hz, 2H), 8.88 (s, 1H), 8.91 (s, 1H); MS (ESI) m/z 477.1 [M+H]⁺.

### Example 16

### (1R,2R)-2-{[4′-({[(4-chlorophenyl)amino]carbonyl}amino)-1,1′-biphenyl-4-yl]carbonyl}cyclopentanecarboxylic acid

Example 16 was prepared using the procedure as described for Example 2 substituting Example 15 for Example 1J. ¹H NMR (500 MHz, DMSO-d₆) δ ppm 1.56-1.85 (m, 4H), 1.98-2.05 (m, 1H), 2.14-2.21 (m, 1H), 3.20-3.25 (m, 1H), 4.07-4.12 (m, 1H), 7.34 (d, J=8.85 Hz, 2H), 7.51 (d, J=8.85 Hz, 2H), 7.60 (d, J=8.55 Hz, 2H), 7.72 (d, J=8.54 Hz, 2H), 7.82 (d, J=8.54 Hz, 2H), 8.06 (d, J=8.54 Hz, 2H), 8.93 (s, 1H), 8.96 (s, 1H), 12.23 (s, 1H); MS (ESI) m/z 463.1 [M+H]⁺.

### Example 17

### methyl (1R,2R)-2-{[4′-({[(4-cyanophenyl)amino]carbonyl}amino)-1,1′-biphenyl-4-yl]carbonyl}cyclopentanecarboxylate

Example 17 was prepared using the procedure as described for Example 1J, substituting 4-cyanophenyl isocyanate for 4-(trifluoromethoxy)phenyl isocyanate. ¹H NMR (500 MHz, DMSO-d₆) δ ppm 1.56-1.87 (m, 4H), 2.00-2.07 (m, 1H), 2.16-2.23 (m, 1H), 3.30-3.34 (m, 1H), 3.58 (s, 3H), 4.09-4.14 (m, 1H), 7.61 (d, J=8.85 Hz, 2H), 7.66 (d, J=8.85 Hz, 2H), 7.73-7.76 (m, 4H), 7.83 (d, J=8.55 Hz, 2H), 8.06 (d, J=8.54 Hz, 2H), 9.08 (s, 1H), 9.28 (s, 1H); MS (DCI) m/z 468.3 [M+H]⁺.

### Example 18

### (1R,2R-2-{[4′-({[(4-cyanophenyl)amino]carbonyl}amino)-1,1′-biphenyl-4-yl]carbonyl}cyclopentanecarboxylic acid

Example 18 was prepared using the procedure as described for Example 2 substituting Example 17 for Example 1J. ¹H NMR (500 MHz, DMSO-d₆) δ ppm 1.59-1.85 (m, 4H), 1.96-2.06 (m, 1H), 2.12-2.22 (m, 1H), 3.22 (dd, J=8.14 Hz, 8.13 Hz, 1H), 4.09 (dd, J=8.90 Hz, 7.67 Hz, 1H), 7.61 (d, J=8.74 Hz, 2H), 7.66 (d, J=8.74 Hz, 2H), 7.72-7.75 (m, 4H), 7.82 (d, J=8.44 Hz, 2H), 8.06 (d, J=8.44 Hz, 2H), 9.05 (s, 1H), 9.26 (s, 1H), 12.24 (br, s, 1H); MS (ESI) m/z 452.3 [M−H]⁺.

### Example 19

### methyl (1R,2R)-2-}[4′-({[(4-methoxyphenyl)amino]carbonyl}amino)-1,1′-biphenyl-4-yl}carbonyl}cyclopentanecarboxylate

Example 19 was prepared using the procedure as described for Example 1J substituting 4-methoxyphenyl isocyanate for 4-(trifuoromethoxy)phenyl isocyanate. ¹H NMR (500 MHz, DMSO-d₆) δ ppm 1.58-1.87 (m, 4H), 2.00-2.07 (m, 1H), 2.16-2.23 (m, 1H), 3.29-3.34 (m, 1H), 3.58 (s, 3H), 3.72 (s, 3H), 4.08-4.13 (m, 1H), 6.88 (d, J=9.15 Hz, 2H), 7.37 (d, J=9.15 Hz, 2H), 7.58 (d, J=8.85 Hz, 2H), 7.71 (d, J=8.85 Hz, 2H), 7.81 (d, J=8.55 Hz, 2H), 8.05 (d, J=8.55 Hz, 2H), 8.54 (s, 1H), 8.79 (s, 1H); MS (DCI) m/z 473.1 [M+H]⁺.

### Example 20

### (1R,2R)-2-{[4′-({[(4-methoxyphenyl)amino]carbonyl}amino)-1,1′-biphenyl-4-yl]carbonyl}cyclopentanecarboxylic acid

Example 20 was prepared using the procedure as described for Example 2 substituting Example 19 for Example 1J. ¹H NMR (500 MHz, DMSO-d₆) δ ppm 1.60-1.91 (m, 4H), 1.95-2.03 (m, 1H), 2.07-2.14 (m, 1H), 3.11-3.17 (m, 1H), 3.72 (s, 3H), 4.01-4.17 (m, 1H), 6.87 (d, J=8.90 Hz, 2H), 7.40 (d, J=8.90 Hz, 2H), 7.60 (d, J=8.90 Hz, 2H), 7.68 (d, J=8.59 Hz, 2H), 7.80 (d, J=8.59 Hz, 2H), 8.11 (d, J=8.59 Hz, 2H), 9.17 (s, 1H), 9.47 (s, 1H); MS (ESI) m/z 459.1 [M+H]⁺.

### Example 21

### methyl (1R,2R)-2-[(4′-{[(isopropylamino)carbonyl]amino}-1,1′-biphenyl-4-yl)carbonyl]cyclopentanecarboxylate

Example 21 was prepared using the procedure as described for Example 1J, substituting isopropyl isocyanate for 4-(trifluoromethoxy)phenyl isocyanate. ¹H NMR (500 MHz, DMSO-d₆) δ ppm 1.11 (d, J=6.45 Hz, 6H), 1.55-1.87 (m, 4H), 1.98-2.06 (m, 1H), 2.15-2.22 (m, 1H), 3.28-3.34 (m, 1H), 3.57 (s, 3H), 3.75-3.82 (m, 1H), 4.07-4.13 (m, 1H), 6.05 (d, J=7.67, 1H), 7.51 (d, J=8.90 Hz, 2H), 7.65 (d, J=8.90 Hz, 2H), 7.79 (d, J=8.59 Hz, 2H), 8.03 (d, J=8.90 Hz, 2H), 8.46 (s, 1H); MS (DCI) m/z 409.1 [M+H]⁺.

### Example 22

### (1R,2R)-2-[(4′-{[(isopropylamino)carbonyl]amino}-1,1′-biphenyl-4-yl)carbonyl]cyclopentanecarboxylic acid

Example 22 was prepared using the procedure as described for Example 2 substituting Example 21 for Example 1J. ¹H NMR (500 MHz, DMSO-d₆) δ ppm 1.10 (d, J=6.44 Hz, 6H), 1.64-2.08 (m, 6H), 3.01-3.07 (m, 1H), 3.72-3.81 (m, 1H), 4.13-4.19 (m, 1H), 6.89 (br s, 1H), 7.54 (d, J=8.59 Hz, 2H), 7.61 (d, J=8.90 Hz, 2H), 7.76 (d, J=8.59 Hz, 2H), 8.11 (d, J=8.29 Hz, 2H), 9.37 (br s, 1H); MS (DCI) m/z 395.1 [M+H]⁺.

### Example 23

### methyl 2-({4′-[({[4-(trifluoromethyl)phenyl]amino}carbonyl)amino]-1,1′-biphenyl-4-yl}carbonyl)cyclohexanecarboxylate

### Example 23A

### methyl 2-(4-bromobenzoyl)cyclohexanecarboxylate

A mixture of 2-(4-bromo-benzoyl)-cyclohexanecarboxylic acid (312 mg, 1 mmol), iodomethane (0.1 mL, 1.5 mmol), and NaHCO₃ (252 mg, 3 mmol) in 8 mL N,N-dimethylformamide was stirred at room temperature under N₂ overnight. LC-MS showed that little starting material remaining. Water (1 mL) was added to the reaction mixture, followed by slow addition of concentrated HCl with stirring at <15° C. to adjust the acidity to pH <7. The reaction mixture was extracted with ethyl acetate (40 mL) and the layers were separated. The aqueous layer was extracted with ethyl acetate (2×30 mL). The combined organic layers were washed with brine, dried over Na₂SO₄, filtered and concentrated. The residue was purified on a flash column eluting with 0-15% ethyl acetate in hexanes to provide the title compound (306 mg, 94% yield). ¹H NMR (500 MHz, DMSO-d₆) δ ppm 1.07-1.50 (m, 4H), 1.70-2.07 (m, 4H), 2.72-2.78 (m, 1H), 3.49 (s, 3H), 3.57-3.66 (m, 1H), 7.75 (d, J=6.75 Hz, 2H), 7.93 (d, J=6.75 Hz, 2H).

### Example 23B

### methyl 2-[(4′-nitro-1,1′-biphenyl-4-yl)carbonyl]cyclohexanecarboxylate

To an ambient slurry of Example 23A (306 mg, 0.94 mmol), 4-nitrophenyl boronic acid (315 mg, 1.88 mmol) and KF (164 mg, 2.82 mmol) in dimethoxyethane/tolutene/ethanol/H₂O (10/1/6/3 ratio, 10 mL) was added palladium tetrakis(triphenylphosphine) (11 mg, 0.0094 mmol) in a single portion. The reaction was heated at 90° C. overnight, cooled to room temperature, filtered through celite, washed with ethyl acetate, concentrated and purified using flash chromatography on SiO₂ column, eluting with 0-15% ethyl acetate in hiexanes to provide the title compound (300 mg, 87% yield). ¹H NMR (500 MHz, DMSO-d₆) δ ppm 1.12-1.23(m, 1H), 1.31-1.53 (m, 3H), 1.72-1.82 (m, 2H), 1.94-1.99 (m, 1H), 2.06-2.09 (m, 1H), 2.76-2.82 (m, 1H), 3.51 (s, 3H), 3.69-3.75 (m, 1H), 7.95 (d, J=8.59 Hz, 2H), 8.05 (d, J=9.21 Hz, 2H), 8.14 (d, J=8 90 Hz, 2H), 8.35 (d, J=8.89 Hz, 2H); MS (ESI) m/z 368.1 [M+H]⁺.

### Example 23C

### methyl 2-[(4′-amino-1,1′-biphenyl-4-yl)carbonyl]cyclohexanecarboxylate

A mixture of Example 23B (300 mg, 0.82 mmol), iron powder (137 mg, 2.5 mmol), and NH₄Cl (44 mg, 0.82 mmol) in a mixture of solvents (20 mL of ethanol and 5 mL of water) was heated at 85° C. under N₂ for 2 h. LC-MS shows reaction finished. The reaction mixture was filtered through celite, washed with ethyl acetate, and concentrated. The residue obtained after concentration was dissolved in ethyl acetate (50 mL), washed with saturated NaHCO₃ solution, brine, dried over Na₂SO₄, and concentrated to provide the title compound, MS (ESI) m/z 338.0 [M+H]⁺.

### Example 23D

### methyl 2-({4′-[({[4-trifluoromethyl)phenyl]amino}carbonyl)amino]-1,1′-biphenyl-4-yl}carbonyl)cyclohexanecarboxylate

A scintillation vial was charged with Example 23C (34 mg, 0.1 mmol), 4-(trifluoromethyl)phenyl isocyanate (19 mg, 0.1 mmol) and tetrahydrofuran (6 mL). It was placed in a shaker at room temperature overnight. LC-MS shows reaction finished The mixture was concentrated and purified on RP-HPLC (Preparative reversed-phase chromatography was performed using a Zorbax SB-C18 7 uM 21.2×250 mm column with UV detection analyzed at 220 and 254 nM. Preparative method: (water with 0.1% trifluoroacetic acid and CH3CN with 0.1% trifluoroacetic acid gradient) 5-95% CH₃CN over 30 minutes at 15 mL/min) to provide the title product. ¹H NMR (500 MHz, DMSO-d₆) δ ppm 1.11-1.56 (m, 4H), 1.72-1.81 (m, 2H) 1.92-2.01 (m, 1H), 2.05-2.08 (m, 1H), 2.74-2.81 (m, 1H), 3.51 (s, 3H), 3.66-3.72 (m, 1H), 7.60-7.75 (m, 8H), 7.82 (d, J=8.90 Hz, 2H), 8.05 (d, J=8.60 Hz, 2H), 8.99 (s, 1H), 9.14 (s, 1H); MS (ESI) m/z 525.2 [M+H]⁺.

### Example 24

### 2-({4′-[({[4-(trifluoromethyl)phenyl]amino}carbonyl)amino]-1,1′-biphenyl-4-yl}carbonyl)cyclohexanecarboxylic acid

A scintillation vial was charged with Example 23D (46 mg, 0.09 mmol), LiOH (19 mg, 0.45 mmol) and 6 mL of 4:1 tetrahydrofuran/water. It was placed in a shaker at r.t. overnight. LC-MS shows reaction finished. The mixture was concentrated and purified on RP-HPLC (Preparative reversed-phase chromatography was performed using a Zorbax SB-C18 7 uM 21.2×250 mm column with UV detection analyzed at 220 and 254 nM. Preparative method: (water with 0.1% trifluoroacetic acid and CH₃CN with 0.1% trifluoroacetic acid gradient) 5-95% CH3CN over 30 minutes at 15 mL/min.) to provide the title product. ¹H NMR (500 MHz, DMSO-d₆) δ ppm 1.15-1.45 (m, 4H), 1.73-1.91 (m, 3H), 2.14-2.20 (m, 1H), 2.61-2.67 (m, 1H), 3.63-3.68 (m, 1H), 7.52-7.60 (m, 6H), 7.75-7.76 (m, 4H), 8.08 (d, J=8.24 Hz, 2H), 10.12 (br s, 2H); MS (ESI) m/z 511.2 [M+H]⁺.

### Example 25

### methyl 2-{[4′-({[(3-chlorophenyl)amino]carbonyl}amino)-1,1′-biphenyl-4-yl]carbonyl}cyclohexanecarboxylate

Example 25 was prepared using the procedure as described for Example 23D, substituting 3-chlorophenyl isocyanate for 4-(trifluoromethyl)phenyl isocyanate. ¹H NMR (500 MHz, DMSO-d₆) δ ppm 1.11-1.55 (m, 4H), 1.72-1.81 (m, 2H), 1.92-1.98 (m, 1H), 2.03-2.08 (m, 1H), 2.75-2.81 (m, 1H), 3.50 (s, 3H), 3.66-3.72 (m, 1H), 7.02-7.04 (m, 1H), 7.27-7.34 (m, 2H), 7.60 (d, J=6.75 Hz, 2H), 7.70-7.74 (m, 3H), 7.81 (d, J=7.06 Hz, 2H), 8.05 (d, J=8.59 Hz, 2H), 8.93 (d, J=2.14 Hz, 2H); MS (ESI) m/z 491.2 [M+H]⁺.

### Example 26

### 2-{[4′-({[(3-chlorophenyl)amino]carbonyl}amino)-1,1′-biphenyl-4-yl]carbonyl}cyclohexanecarboxylic acid

Example 26 was prepared using the procedure as described for Example 24 substituting Example 25 for Example 23D. ¹H NMR (500 MHz, DMSO-d₆) δ ppm 1.10-1.46 (m, 4H), 1.72-1.93 (m, 3H), 2.07-2.12 (m, 1H), 2.65-2.70 (m, 1H), 3.60-3.66 (m, 1H), 7.01-7.02 (m, 1H), 7.28-7.31 (m, 2H), 7.57 (d, J=8.54 Hz, 2H), 7.66-7.68 (m, 2H), 7.74-7.80 (m, 3H), 8.05 (d, J=8.54 Hz, 2H), 9.19 (br s, 2H), 12.09 (br s, 1H); MS (ESI) m/z 477.2 [M+H]⁺.

### Example 27

### (1R,2R)-2-((R)-hydroxy{4′-[({[4-(trifluoromethyl)phenyl]amino}carbonyl)amino]-1,1′-biphenyl-4-yl}methyl)cyclopentanecarboxylic acid and (1R,2R)-2-((S)-hydroxy{4′-[({[4-(trifluoromethyl)phenyl]amino}carbonyl)amino]-1,1′-biphenyl-4-yl}methyl)cyclopentanecarboxylic acid

Into a 25 ml round bottom flask, Example 4 (25 mg, 0.05 mmol) and methanol (6 mL) were added. The reaction mixture was treated with NaBH₄ (8 mg, 2 mmol), stirred at room temperature under N₂ overnight, quenched with 1N HCT solution, and extracted with ethyl acetate (40 mL). The aqueous layer was extracted with ethyl acetate (40 mL), and the combined organic layers were dried over Na2SO₄, filtered and concentrated. The residue was purified on RP-HPLC (Preparative reversed-phase chromatography was performed using a Zorbax SB-C18 7 uM 21.2×250 mm column with UV detection analyzed at 220 and 254 nM. Preparative method: (water with 0.1% trifluoroacetic acid and CH₃CN with 0.1% trifluoroacetic acid gradient) 5-95% CH3CN over 30 minutes at 15 mL/min.) to provide two diastereomers. The diastereomers were injected onto an analytical RP-HPLC system (Agilent ZORBAX SB-C18 column 5 um, 4.6×250 mm, solvent flow: 1.5 ml/min, stop time: 20 min, beginning with 100% H₂O (with 0.1% trifluoroacetic acid) for 0-1 min, 1-15 min: 0% CH₃CN to 100% CH₃CN, 15-18 min: 100% CH₃CN, 18-19 min: 100% to 0% CH₃CN, 19-20 min. 100% H₂O (with 0.1% trifluoroacetic acid)). The diastereomer with retention time of 11.35 min is characterized as follows: ¹H NMR (500 MHz, DMSO-d₆) δ ppm 1.45-1.72 (m, 5H), 1.82-1.94 (m, 1H), 2.41-2.47 (m, 1H), 2.61-2.67 (m, 1H), 4.62 (d, J=491 Hz, 1H), 5.21 (br, s, 1H), 7.35 (d, J=7.98 Hz, 2H), 7.54-7.69 (m, 10H), 8.91 (s, 1H), 9.14 (s, 1H), 11.86 (br S, 1H); MS (ESI) m/z 497.3 [M−H]⁺. The diastereomer with retention time of 11.72 min is characterized as follows: ¹H NMR (500 MHz, DMSO-d₆) δ ppm 1.23-1.31 (m, 1H) 1.42-1.57 (m, 3H), 1.63-1.73 (m, 1H), 1.84-1.94 (m, 1H), 2.52-2.57 (m, 1H), 2.62-2.69 (m, 1H), 4.40 (d, J=7.67 Hz, 1H), 5.38 (br, s, 1H), 7.37 (d, J=7.98 Hz, 2H), 7.54-7.69 (m, 10H), 8.90 (s, 1H), 9.13 (s, 1H), 11.79 (br s, 1H); MS (ESI) m/z 497.5 [M−H]⁺.

### Example 28

### methyl (1R,2R)-2-{[4′-({[(4-fluorophenyl)amino]carbonyl}amino)-1,1′-biphenyl-4-yl]carbonyl}cyclopentanecarboxylate

Example 28 was prepared using the procedure as described for Example 1J, substituting 4-fluorophenyl isocyanate for 4-(trifluoromethoxy)phenyl isocyanate. ¹H NMR (500 MHz, DMSO-d₆) δ ppm 1.56-1.87 (m, 4H), 2.00-2.07 (m, 1H), 2.16-2.24 (m,1H), 3.29-3.34 (m, 1H), 3.58 (s, 3H), 4.09-4.14 (m, 1H), 7.13 (t, J=8.85 Hz, 2H), 7.47-7.50 (m, 2H), 7.59 (d, J=8.54 Hz, 2H), 7.72 (d, J'8.85 Hz, 2H), 7.82 (d, J=8.55 Hz, 2H), 8.06 (d, J=8.24 Hz, 2H), 8.76 (s, 1H), 8.86 (s, 1H); MS (ESI) m/z 461.2 [M+H]⁺.

### Example 29

### (1R,2R)-2-{[4′-({[(4-fluorophenyl)amino]carbonyl}amino)-1,1′-biphenyl-4-yl]carbonyl}cyclopentanecarboxylic acid

Example 29 was prepared using the procedure as described for Example 2 substituting Example 28 for Example 1J. ¹H NMR (500 MHz, DMSO-d₆) δ ppm 1.58-1.87 (m, 4H), 1.97-2.06 (m, 1H), 2.13-2.22 (m, 1H), 3.22 (dd, J=8.13 Hz, 8.13 Hz, 1H), 4.09 (dd, J=8.90 Hz, 7.82 Hz, 1H), 7.13 (t, J=8.89 Hz, 2H), 7.46-7.50 (m,2H), 7.59 (d, J=8.75 Hz, 2H), 7.71 (d, J=8.74 Hz, 2H), 7.82 (d, J=8.44 Hz, 2H), 8.06 (d, J=8.59 Hz, 2H), 8.76 (s, 1H), 8.86 (s, 1H), 12.19 (br, s, 1H); MS (ESI) m/z 447.2 [M+H]⁺.

### Example 30

### methyl (1R,2R)-2-{[4′-({[(3-fluorophenyl)amino]carbonyl}amino)-1,1′-biphenyl-4-yl]carbonyl}cyclopentanecarboxylate

Example 30 was prepared using the procedure as described for Example 1J substituting 3-fluorophenyl isocyanate for 4-(trifluoromethoxy)phenyl isocyanate. ¹H NMR (500 MHz, DMSO-d₆) δ ppm 1.57-1.87 (m, 4H), 2.00-2.07 (m, 1H), 2.16-2.23 (m, 1H), 3.30-3.35 (m, 1H), 3.58 (s, 3H), 4.09-4.14 (m, 1H), 6.78-6.82 (m, 1H), 7.14 (d, J=8.24 Hz, 1H), 7.31 (dd, J=7.02 Hz, 8.24 Hz, 1H), 7.49-7.52 (m, 1H), 7.60 (d, J=8.54 Hz, 2H), 7.73 (d, J=8.54 Hz, 2H), 7.82 (d, J=8.54 Hz, 2H), 8.06 (d, J=8.54 Hz, 2H), 8.94 (s, 1H), 8.97 (s, 1H); MS (ESI) m/z 461.1 [M+H]⁺.

### Example 31

### (1R,2R)-2-{[4′-({[(3-fluorophenyl)amino]carbonyl}amino)-1,1′-biphenyl-4-yl]carbonyl}cyclopentanecarboxylic acid

Example 31 was prepared using the procedure as described for Example 2 substituting Example 30 for Example 1J. ¹H NMR (500 MHz, DMSO-d₆) δ ppm 1.58-1.85 (m, 4H), 1.97-2.07 (m, 1H), 2.13-2.20 (m, 1H), 3.22 (dd, J=7.98 Hz, 7.98 Hz, 1H), 4.09 (dd, J=9.05 Hz, 7.67 Hz, 1H), 6.77-6.82 (m, 1H), 7.14 (d, J=9.20 Hz, 1H), 7.31 (dd, J=7.06Hz, 8.13 Hz, 1H), 7.48-7.52 (m, 1H), 7.60 (d, J=8.74 Hz, 2H), 7.72 (d, J=7.74 Hz, 2H), 7.82 (d, J=8.59 Hz, 2H), 8.06 (d, J=8.44 Hz, 2H), 8.92 (s, 1H), 8.95 (s, 1H), 12.20 (br, s, 1H); MS (ESI) m/z 447.1 [M+H]⁺.

### Example 32

### methyl (1R,2R)-2-{[4′-({[(2-fluorophenyl)amino]carbonyl}amino)-1,1′-biphenyl-4-yl]carbonyl}cyclopentanecarboxylate

Example 32 was prepared using the procedure as described for Example 1J substituting 2-fluorophenyl isocyanate for 4-(trifluoromethoxy)phenyl isocyanate. ¹H NMR (500 MHz, DMSO-d₆) δ ppm 1.57-1.87 (m, 4H), 2.00-2.07 (m, 1H), 2.16-2.22 (m, 1H), 3.30-3.35 (m, 1H), 3.58 (s, 3H), 4.09-4.14 (m, 1H), 7.00-7.05 (m, 1H), 7.16 (t, J=7.63 Hz, 1H), 7.23-7.27 (m, 1H), 7.60 (d, J=8.85 Hz, 2H), 7.73 (d, J=8.54 Hz, 2H), 7.82 (d, J=8.55 Hz, 2H), 8.06 (d, J=8.55 Hz, 2H), 8.15-8.18 (m, 1H), 8.60 (br, s, 1H), 9.26 (s, 1H); MS (ESI) m/z 461.1 [M+H]⁺.

### Example 33

### (1R,2R)-2-{[4′-({[(2-fluorophenyl)amino]carbonyl}amino)-1,1′-biphenyl-4-yl]carbonyl}cyclopentanecarboxylic acid

Example 33 was prepared using the procedure as described for Example 2 substituting Example 32 for Example 1J. ¹H NMR (500 MHz, DMSO-d₆) δ ppm 1.59-1.84 (m, 4H), 1.95-2.01 (m, 1H), 2.09-2.16 (m, 1H), 3.14-3.18 (m, 1H), 4.10-4.15 (m, 1H), 7.01-7.05 (m, 1H), 7.15 (t, J=8.24 Hz, 1H), 7.22-7.26 (m, 1H), 7.61 (d, J=8.54 Hz, 2H), 7.72 (d, J=8.54 Hz, 2H), 7.82 (d, J=8.24 Hz, 2H), 8.08-8.13 (m, 3H), 8.91 (br, s, 1H), 9.59 (br, s, 1H); MS (ESI) m/z 447.2 [M+H]⁺.

### Example 34

### methyl 2-{[4′-({[(4-fluorophenyl)amino]carbonyl}amino)-1,1′-biphenyl-4-yl]carbonyl}cyclohexanecarboxylate

Example 34 was prepared using the procedure as described for Example 23D substituting 4-fluorophenyl isocyanate for 4-(trifluoromethyl)phenyl isocyan ate. ¹H NMR (500 MHz, DMSO-d₆) δ ppm 1.09-1.52 (m, 4H), 1.72-1.81 (m, 2H), 1.94-2.07 (m, 2H), 2.71-2.80 (m, 1H), 3.50 (s, 3H), 3.65-3.72 (m, 1H), 7.13 (t, J=8.90 Hz, 2H), 7.46-7.50 (m, 2H), 7.59 (d, J=8.59 Hz, 2H), 7.71 (d, J=8.59 Hz, 2H), 7.81 (d, J=8.59 Hz, 2H), 8.05 (d, J=8.59 Hz, 2H), 8.74 (s, 1H), 8.84 (s, 1H); MS (ESI) m/z 475.2 [M+H]⁺.

### Example 35

### 2-{[4′-({[(4-fluorophenyl)amino]carbonyl}amino)-1,1′-biphenyl-4-yl]carbonyl}cyclohexanecarboxylic acid

Example 35 was prepared using the procedure as described for Example 24 substituting Example 34 for Example 23D. ¹H NMR (500 MHz, DMSO-d₆) δ ppm 1.08-1.53 (m, 4H), 1.72-1.82 (m, 2H), 1.92-2.10 (m, 2H), 2.67-2.72 (m, 1H), 3.59-3.65 (m, 1H), 7.13 (t, J=8.90 Hz, 2H), 7.47-7.50 (m, 2H), 7.59 (d, J=8.59 Hz, 2H), 7.70 (d, J=8.90 Hz, 2H), 7.80 (d, J=8.59 Hz, 2H), 8.05 (d, J=8.28 Hz, 2H), 8.76 (s, 1H), 8.85 (s, 1H), 11.98 (br s, 1H); MS (ESI) m/z 461.2 [M+H]⁺.

### Example 36

### methyl (1R,2R)-2-({4′-[({[3-(trifluoromethyl)phenyl]amino}carbonyl)amino]-1,1′-biphenyl-4-yl}carbonyl)cyclopentanecarboxylate

Example 36 was prepared using the procedure as described for Example 1J substituting 3-(trifluoromethyl)phenyl isocyanate for 4-(trifluoromethoxy)phenyl isocyanate. ¹H NMR (500 MHz, DMSO-d₆) δ ppm 1.57-1.87 (m, 4H), 2.00-2.08 (m, 1H), 2.16-2.24 (m, 1H), 3.30-3.35 (m, 1H), 3.58 (s, 3H), 4.08-4.14 (m, 1H), 7.32 (d, J=7.68 Hz, 1H), 7.53 (t, J=7.98 Hz, 1H), 7.60-7.63 (m, 3H), 7.73 (d, J=8.28 Hz, 2H), 7.83 (d, J=7.79 Hz, 2H), 8.03-8.07 (m, 3H), 9.01 (s, 1H), 9.12 (s, 1H); MS (ESI) m/z 511.2 [M+H]⁺.

### Example 37

### (1R,2R)-2-({4′-[({[3-(trifluoromethyl)phenyl]amino}carbonyl)amino]-1,1′-biphenyl-4-yl}carbonyl)cyclopentanecarboxylic acid

Example 37 was prepared using the procedure as described for Example 2 substituting Example 36 for Example 1J. ¹H MAR (500 MHz, DMSO-d₆) δ ppm 1.55-1.87 (m, 4H), 1.98-2.05 (m, 1H), 2.14-2.21(m, 1H), 3.20-3.24 (m, 1H), 4.07-4.12 (m, 1H), 7.33 (d, J=7.63 Hz, 1H), 7.53 (t, J=7.93 Hz, 1H), 7.60-7.62 (m, 3H), 7.73 (d, J=8.54 Hz, 2H), 7.82 (d, J=8.54 Hz, 2H), 8.04-8.08 (m, 3H), 9.06 (s, 1H), 9.17 (s, 1H), 12.22 (br s, 1H); MS (ESI) m/z 497.2 [M+H⁺.

### Example 38

### methyl 2-({4′-[({[3-(trifluoromethyl)phenyl]amino}carbonyl)amino]-1,1′-biphenyl-4-yl}carbonyl)cyclohexanecarboxylate

Example 38 was prepared using the procedure as described for Example 23D substituting 3-(trifluoromethyl)phenyl isocyanate for 4-(trifluoromethyl)phenyl isocyanate. ¹H NMR (500 MHz, DMSO-d₆) δ ppm 1.11-1.56 (m, 4H), 1.72-1.82 (m, 2H), 1.94-1.98 (m, 1H), 2.05-2.08 (m, 1H), 2.74-2.81 (m, 1H), 3.50 (s, 3H), 3.66-3.72 (m, 1H), 7.32 (d, J=7.67 Hz, 1H), 7.53 (t, J=7.98 Hz, 1H), 7.59-7.63 (m, 3H), 7.72 (d, J=8.59 Hz, 2H), 7.82 (d, J=8.59 Hz, 2H), 8.03-8.06 (m, 3H), 8.98 (s, 1H), 9.09 (s, 1H); MS (ESI) m/z 523.2 [M−H]⁺.

### Example 39

### 2-({4′-[({[3-(trifluoromethyl)phenyl]amino}carbonyl)amino]-1,1′-biphenyl-4-yl}carbonyl)cyclohexanecarboxylic acid

Example 39 was prepared using the procedure as described for Example 24 substituting Example 38 for Example 23D. ¹H NMR (500 MHz, DMSO-d₆) δ ppm 1.09-1.53 (m, 4H), 1.72-1.85 (m, 2H), 1.92-1.95 (m, 1H), 2.07-2.10 (m, 1H), 2.66-2.72 (m, 1H), 3.59-3.66 (m, 1H), 7.32 (d, J=7.36 Hz, 1H), 7.53 (t, J=7.97 Hz, 1H), 7.59-7.62 (m, 3H), 7.72 (d, J=8.90 Hz, 2H), 7.81 (d, J=8.59 Hz, 2H), 8.03-8.06 (m, 3H), 8.98 (s, 1H), 9.09 (s, 1H), 12.07 (br s, 1H); MS (ESI) m/z 511.2 [M−H]⁺.

### Example 40

### methyl (1R,2R)-2-({4′-[({[2-(trifluoromethyl)phenyl]amino}carbonyl)amino]-1,1′-biphenyl-4-yl}carbonyl)cyclopentanecarboxylate

Example 40 was prepared using the procedure as described for Example 1J substituting 2-(trifluoromethyl)phenyl isocyanate for 4-(trifluoromethoxy)phenyl isocyanate. ¹H NMR (500 MHz, DMSO-d6) δ ppm 1.56-1.87 (m, 4H), 2.00-2.08 (m, 1H), 2.17-2.24 (m, 1H), 3.29-3.35 (m, 1H), 3.58 (s, 3H), 4.08-4.14 (m, 1H), 7.30 (t, J=7.37 Hz, 1H), 7.59-7.75 (m, 6H), 7.83 (d, J=8.59 Hz, 2H), 7.96 (d, J=8.29 Hz, 1H), 8.06 (d, J=8.29 Hz, 2H), 8.13 (s, 1H), 9.54 (s, 1H); MS (ESI) m/z 511.2 [M+H]⁺.

### Example 41

### (1R,2R)-2-({4′-[({[2-(trifluoromethyl)phenyl]amino}carbonyl)amino]-1,1′-biphenyl-4-yl}carbonyl)cyclopentanecarboxylic acid

Example 41 was prepared using the procedure as described for Example 2 substituting Example 40 for Example 1J. ¹H NMR (500 MHz, DMSO-d₆) δ ppm 1.56-1.87 (m, 4H), 1.97-2.06 (m, 1H), 2.14-2.20 (m, 1H), 3.19-3.25 (m, 1H), 4.06-4.12 (m, 1H), 7.30 (t, J=7.67 Hz, 1H), 7.59-7.75 (m, 6H), 7.82 (d, J=6.75 Hz, 2H), 7.96 (d, J=8.29 Hz, 1H), 8.06 (d, J=6.75 Hz, 2H), 8.13 (s, 1H), 9.54 (s, 1H), 12.20 (br s, 1H); MS (ESI) m/z 497.2 [M+H]⁺.

### Example 42

### methyl 2-({4′-[({[2-(trifluoromethyl)phenyl]amino}carbonyl)amino]-1,1′-biphenyl-4-yl}carbonyl)cyclohexanecarboxylate

Example 42 was prepared using the procedure as described for Example 23D substituting 2-(trifluoromethyl)phenyl isocyanate for 4-(trifluoromethyl)phenyl isocyanate. ¹H NMR (500 MHz, DMSO-d₆) δ ppm 1.11-1.54 (m, 4H), 1.72-1.81 (m, 2H), 1.91-1.97 (m, 1H), 2.05-2.08 (m, 1H), 2.75-2.80 (m, 1H), 3.50 (s, 3H), 3.65-3.72 (m, 1H), 7.30 (t, J=7.67 Hz, 1H), 7.60-7.74 (m, 6H), 7.82 (d, J=8.59 Hz, 2H), 7.96 (d, J=8.29 Hz, 1H), 8.05 (d, J=8.59 Hz, 2H), 8.13 (s, 1H), 9.54 (s, 1H); MS (ESI) m/z 525.3 [M+H]⁺.

### Example 43

### 2-({4′-[({[2-(trifluoromethyl)phenyl]amino}carbonyl)amino]-1,1′-biphenyl-4-yl}carbonyl)cyclohexanecarboxylic acid

Example 43 was prepared using the procedure as described for Example 24 substituting Example 42 for Example 23D. ¹H NMR (500 MHz, DMSO-d₆) δ ppm 1.07-1.53 (m, 4H), 1.69-1.82 (m, 2H), 1.91-1.95 (m, 1H), 2.06-2.10 (m, 1H), 2.67-2.72 (m, 1H), 3.59-3.66 (m, 1H), 7.30 (t, J=7.67 Hz, 1H), 7.59-7.74 (m, 6H), 7.81 (d, J=8.59 Hz, 2H), 7.96 (d, J=8.29 Hz, 1H), 8.06 (d, J=8.59 Hz, 2H), 8.13 (s, 1H), 9.54 (s, 1H), 12.04 (br s, 1H); MS (ESI) m/z 511.2 [M+H]⁺.

### Example 44

### (1R,2R)-2-({4′-[(anilinocarbonyl)amino]-1,1′-biphenyl-4-yl}carbonyl)cyclopentanecarboxamide

Example 10 (0.12 g, 0.28 mmol) was added to a mixture of N-hydroxy succinamide (0.065 g, 0.56 mmol), N-(3-dimethylaminopropyl)-N′-ethylcarbodiimide hydrochloride (0.107 g, 0.56 mmol) and N-methyl morpholine (0.16 mL, 1.12 mmol) in dichloromethane (5 mL) and stirred at room temperature for 2 h. The solvents were removed on a rotary evaporator and then diluted with a 1:1 mixture of ethyl acetate and water (20 mL). The organic layers were separated, washed with brine, dried (MgSO₄), filtered and concentrated to a white solid. The residue was taken up in dioxane (5 mL) and ammonium hydroxide (0.2 mL, 1.4 mmol) was added to the solution at r.t. and stirred for 1 h. The reaction mixture was filtered and the filtrate concentrated to a white solid that was purified by RP-HPLC (preparative reversed-phase chromatography was performed using a Zorbax SB-C18 7 uM 21.2×250 mm column with UV detection analyzed at 220 and 254 nM. Preparative method: (water with 0.1% trifluoroacetic acid and CH₃CN with 0.1% trifluoroacetic acid gradient) 5-95% CH3CN over 30 minutes at 15 mL/min.) to afford the title compound. ¹H NMR (500 MHz, DMSO-d₆) δ ppm 1.56-1.79 (m, 4H), 1.98-2.04 (m, 1H), 2.07-2.11 (m, 1H), 3.01-3.06 (m, 1H), 4.07-4.13 (m, 1H), 6.78 (s, 1H), 6.98 (t, 1H, 7.36 Hz), 7.27-7.31 (m, 3H), 7.47 (d, J=7.67 Hz, 2H), 7.59 (d, J=8.90 Hz, 2H), 7.71 (d, J=8.90 Hz, 2H), 7.80 (d, J=8.59 Hz, 2H), 8.02 (d, J=8.59 Hz, 2H), 8.72 (s, 1H), 8.86 (s, 1H); MS (ESI) m/z 428.1 [M+H]⁺.

### Example 45

### (1R,2R)-2-({4′-[(anilinocarbonyl)amino]-1,1′-biphenyl-4-yl}carbonyl)-N-methylcyclopentanecarboxamide

A mixture of Example 10 (24 mg, 0.056 mmol), methyl amine (2M in tetrahydrofuran solution, 0.1 mL, 0.067 mmol), 1-ethyl-(3-dimethylaminopropyl)carbodiimide hydrochloride (13 mg, 0.067 mmol), 1-hydroxybenzotriazole hydrate (9 mg, 0.067 mmol), and N-methyl morpholine (0.14 mg, 0.13 mmol) in N,N-dimethylformamide (5 mL) was stirred at room temperature overnight. The solvents were removed on a rotary evaporator and the residue diluted with a 1 :1 mixture of ethyl acetate and water (20 mL). The organic layers were separated, washed with brine, dried (MgSO₄), filtered and concentrated. The residue was purified by RP-HPLC (Preparative reversed-phase chromatography was performed using a Zorbax SB-C18 7 uM 21.2×250 mm column with UV detection analyzed at 220 and 254 nM. Preparative method: (water with 0.1% trifluoroacetic acid and CH₃CN with 0.1% trifluoroacetic acid gradient) 5-95% CH3CN over 30 minutes at 15 mL/min.) to afford the title compound. ¹H NMR (500 MHz, DMSO-d₆) δ ppm 1.60-1.80 (m, 4H), 1.94-2.00 (m, 1H), 2.08-2.15 (m, 1H), 2.53 (d, J=4.58 Hz, 3H), 2.98-3.03 (m, 1H), 4.06-4.11 (m, 1H), 6.98 (t, J=7.63 Hz, 1H), 7.29 (t, J=7.63 Hz, 2H), 7.47 (d, J=7.63 Hz, 2H), 7.60 (d, J=8.55 Hz, 2H), 7.71 (d, J=8.84 Hz, 2H), 7.77-7.81 (m, 3H), 8.00 (d, J=8.54 Hz, 2H), 8.76 (s, 1H), 8.90 (s, 1H); MS (ESI) m/z 442.2 [M+H]⁺.

### Example 46

### methyl (1R,2R)-2-{[4′-({[(2,6-diisopropylphenyl)amino]carbonyl}amino)-1,1′-biphenyl-4-yl]carbonyl}cyclopentanecarboxylate

Example 46 was prepared using the procedure as described for Example 1J, substituting 2,6-diisopropylphenyl isocyanate for 4-(trifluoromethoxy)phenyl isocyanate. ¹H NMR (500 MHz, DMSO-d₆) δ ppm 1.17 (d, J=6.71 Hz, 12H), 1.56-1.87 (m, 4H), 2.00-2.06 (m, 1H), 2.16-2.22 (m, 1H), 3.15-3.20 (m, 2H), 3.29-3.34 (m, 1H), 3.58 (s, 3H), 4.08-4.13 (m, 1H), 7.17 (d, J=7.63 Hz, 2H), 7.26 (t, J=7.94 Hz, 1H), 7.58 (d, J=8.54 Hz, 2H), 7.69-7.72 (m, 3H), 7.80 (d, J=8.54 Hz, 2H), 8.05 (d, J=8.54 Hz, 2H), 8.97 (br s, 1H); MS (ESI) m/z 527.4 [M+H]⁺.

### Example 47

### (1R,2R)-2-{[4′-({[(2,6-diisopropylphenyl)amino}carbonyl}amino)-1,1′-biphenyl-4-yl]carbonyl}cyclopentanecarboxylic acid

Example 47 was prepared using the procedure as described for Example 2 substituting Example 46 for Example 1J. ¹H NMR (500 MHz, DMSO-d₆) δ ppm 1.17 (d, J=6.75 Hz, 12H), 1.55-1.87 (m, 4H), 1.97-2.05 (m, 1H), 2.13-2.19 (m, 1H), 3.16-3.25 (m, 3H), 4.05-4.12 (m, 1H), 7.17 (d, J=7.67 Hz, 2H), 7.26 (t, J=7.37 Hz, 1H), 7.58 (d, J=8.90 Hz, 2H), 7.68-7.71 (m, 3H), 7.80 (d, J=8.59 Hz, 2H), 8.05 (d, J=8.59 Hz, 2H), 8.94 (br s, 1H), 12.19 (br s, 1H); MS (ESI) m/z 513.3 [M+H]⁺.

### Example 48

### methyl (1R,2R)-2-{[4′-({[(2,6-dimethylphenyl)amino]carbonyl}amino)-1,1′-biphenyl-4-yl]carbonyl}cyclopentanecarboxylate

Example 48 was prepared using the procedure as described for Example 1J substituting 2,6-dimethylphenyl isocyanate for 4-(trifluoromethoxy)phenyl isocyanate. ¹H NMR (500 MHz, DMSO-d₆) δ ppm 1.56-1.87 (m, 4H), 2.00-2.07 (m, 1H), 2.16-2.22 (m, 7H), 3.29-3.34 (m, 1H), 3.58 (s, 3H), 4.08-4.13 (m, 1H), 7.05-7.10 (m, 3H), 7.59 (d, J=8.85 Hz, 2H), 7.70 (d, J=8.85 Hz, 2H), 7.79-7.82 (m, 3H), 8.05 (d, J=8.54 Hz, 2H), 8.95 (s, 1H); MS (ESI) m/z 471.3 [M+H]⁺.

### Example 49

### (1R,2R)-2-{[4′-({[(2,6-dimethylphenyl)amino]carbonyl}amino)-1,1′-biphenyl-4-yl]carbonyl}cyclopentanecarboxylic acid

Example 49 was prepared using the procedure as described for Example 2 substituting Example 48 for Example 1J. ¹H NMR (500 MHz, DMSO-d₆) δ ppm 1.59-1.85 (m, 4H), 1.96-2.06 (m, 1H), 2.13-2.20 (m, 1H), 2.23 (s, 6H), 3.22 (dd, J=8.28 Hz, 7.98 Hz, 1H), 4.09 (dd, J=7.67 Hz, 7.68 Hz, 1H), 7.05-7.10 (m, 3H), 7.59 (d, J=8.90 Hz, 2H), 7.69 (d, J=8.90 Hz, 2H), 7.78-7.81 (m, 3H), 8.05 (d, J=8.59 Hz, 2H), 8.93 (s, 1H); MS (ESI) m/z 457.2 [M+]⁺.

### Example 50

### methyl (1R,2R)-2-({4′-[({[2-fluoro-5-(trifluoromethyl)phenyl]amino}-1,1′-biphenyl-4-yl}carbonyl)cyclopentanecarboxylate

Example 50 was prepared using the procedure as described for Example 1J substituting (2-fluoro-5-trifluoromethyl)phenyl isocyanate for 4-(trifluoromethoxy)phenyl isocyanate. ¹H NMR (500 MHz, DMSO-d₆) δ ppm 1.58-1.86 (m, 4H), 2.01-2.07 (m, 1H), 2.17-2.23 (m, 1H), 3.30-3.35 (m 1H), 3.58 (s, 3H), 4.09-4.14 (m, 1H), 7.39-7.42 (m, 1H), 7.49-7.55 (m 1H), 7.61 (d, J=8.84 Hz, 2H), 7.75 (d, J=8.85 Hz, 2H), 7.83 (d, J=8.54 Hz, 2H), 8.06 (d, J=8.54 Hz, 2H), 8.63 (dd, J=5.19 Hz, 2.14 Hz, 1H), 8.95 (d, J=2.75 Hz, 1H), 9.36 (s, 1H); MS (ESI) m/z 529.2 [M+H]⁺.

### Example 51

### (1R,2R)-2-({4′-[({[2-fluoro-5-(trifluoromethyl)phenyl]amino}carbonyl)amino]-1,1′-biphenyl-4-yl}carbonyl)cyclopentanecarboxylic acid

Example 49 was prepared using the procedure as described for Example 2 substituting Example 50 for Example 1J. ¹H NMR (500 MHz, DMSO-d₆) δ ppm 1.58-1.86 (m, 4H), 1.98-2.04 (m, 1H), 2.13-2.17 (m, 1H), 3.20-3.26 (m 1H), 4.06-4.12 (m, 1H), 7.40-7.42 (m, 1H), 7.48-7.53 (m 1H), 7.61 (d, J=8.90 Hz, 2H), 7.74 (d, J'8.90 Hz, 2H), 7.83 (d, J=8.60 Hz, 2H), 8.06 (d, J=8.60 Hz, 2H}, 8.63 (dd, J=4.61 Hz, 2.15 Hz, 1H), 8.94 (d, J=2.76 Hz, 1H), 9.36 (s, 1H), 12.17 (br s, 1H); MS (EST) m/z 515.2 [M+H]⁺.

### Example 52

### methyl (1R,2R)-2-[(4′-{[(thien-2-ylamino)carbonyl]amino}-1,1′-biphenyl-4-yl)carbonyl]cyclopentanecarboxylate

Example 52 was prepared using the procedure as described for Example 1J substituting 2-thienyl isocyanate for 4-(trifluoromethoxy)phenyl isocyanate. ¹H NMR (500 MHz, DMSO-d₆) δ ppm 1.58-1.87 (m, 4H), 2.00-2.06 (m, 1H), 2.16-2.22 (m, 1H), 3.30-3.35 (m, 1H), 3.58 (s, 3H), 4.09-4.14 (m, 1H), 6.58-6.59 (m, 1H), 6.82-6.83 (m, 1H), 6.89-6.90 (m, 1H), 7.60 (d, J=8.54 Hz, 2H), 7.72 (d, J=8.84 Hz, 2H), 7.82 (d, J=8.54 Hz, 2H), 8.06 (d, J=8.54 Hz, 2H), 8.93 (s, 1H), 9,69 (s, 1H); MS (ESI) m/z 449.2 [M+H]⁺.

### Example 53

### (1R,2R)-2-[(4′-{[(thien-2-ylamino)carbonyl]amino}-1,1′-biphenyl-4-yl)carbonyl]cyclopentanecarboxylic acid

Example 53 was prepared using the procedure as described for Example 2 substituting Example 52 for Example 1J. ¹H NMR (500 MHz, DMSO-d₆) δ ppm 1.56-1.87 (m, 4H), 1.97-2.06 (m, 1H), 2.13-2.21 (m, 1H), 3.17-3.25 (m, 1H), 4.06-4.12 (m, 1H), 6.58 (d, J=3.68 Hz, 1H), 6.81-6.83 (m, 1H), 6.88 (d, J=6.76 Hz, 1H), 7.60 (d, J=8.59 Hz, 2H), 7.72 (d, J=8.59 Hz, 2H), 7.82 (d, J=8.29 Hz, 2H), 8.06 (d, J=8.28 Hz, 2H), 9.04 (s, 1H), 9.81 (s, 1H), 12.19 (br s, 1H); MS (ESI) m/z 435.1 [M+H]⁺.

### Example 54

### methyl (1R²R)-2-[(4′-{[(pyridin-3-ylamino)carbonyl]amino}-1,1′-biphenyl-4-yl)carbonyl]cyclopentanecarboxylate

Example 54 was prepared using the procedure as described for Example 1J substituting pyridine-3-isocyanate for 4-(trifluoromethoxy)phenyl isocyanate. ¹H NMR (500 MHz, DMSO-d₆) δ ppm 1.56-1.87 (m, 4H), 2.01-2.08 (m, 1H), 2.15-2.23 (m, 1H), 3.30-3.35 (m, 1H), 3.58 (s, 3H), 4.07-4.14 (m, 1H), 7.64 (d, J=8.85 Hz, 2H), 7.71-7.76 (m, 3H), 7.83 (d, J=8.55 Hz, 2H), 8.06 (d, J=8.54 Hz, 2H), 8.22 (d, J=8.54 Hz, 1H), 8.40 (d, J=4.58 1H), 8.95 (d, J=2.14 Hz, 1H), 9.43 (s, 1H), 9.61 (s, 1H); MS (ESI) m/z 444.2 [M+H⁺.

### Example 55

### (1R,2R)-2-[(4′-{[(pyridin-3-ylamino)carbonyl]amino}-1,1′-biphenyl-4-yl)carbonyl]cyclopentanecarboxylic acid

Example 55 was prepared using the procedure as described for Example 2 substituting Example 54 for Example 1J. ¹H NMR (500 MHz, DMSO-d₆) δ ppm 1.56-1.86 (m, 4H), 1.98-2.06 (m, 1H), 2.14-2.21 (m, 1H), 3.20-3.25 (m, 1H), 4.07-4.12 (m, 1H), 7.40 (dd, J=4.88 Hz, 3.66 Hz, 1H), 7.62 (d, J=8.54 Hz, 2H), 7.73 (d, J=8.54 Hz, 2H), 7.82 (d, J=8.24 Hz, 2H), 8.00-8.02 (m, 1H), 8.07 (d, J=8.55 Hz, 2H), 8.25 (d, J=3.66 Hz, 1H), 8.69 (s, 1H), 9.03 (s, 1H), 9.08 (s, 1H), 12.23 (br s, 1H); MS (ESI) m/z 430.1 [M+H]⁺.

### Example 56

### methyl (1R,2R)-2-[(4′-{[(cyclohexylamino)carbonyl]amino}-1,1′-biphenyl-4-yl)carbonyl]cyclopentanecarboxylate

A scintillation vial was charged with the Example 1I (20 mg, 0.06 mmol), cyclohexyl isocyanate (9 mg, 0.07 mmol) and tetrahydrofuran (6 mL). It was placed in a shaker at room temperature overnight. The mixture was concentrated and purified on a RP-HPLC (Preparative reversed-phase chromatography was performed using a Zorbax SB-C18 7 uM 21.2×250 mm column with UV detection analyzed at 220 and 254 nM. Preparative method: (water with 0.1% trifluoroacetic acid and CH₃CN with 0.1% trifluoroacetic acid gradient) 5-95% CH₃CN over 30 minutes at 15 mL/min.) to provide the title product. ¹H NMR (500 MHz, DMSO-d₆) δ ppm 1.14-1.37 (m, 5H), 1.52-1.89 (m, 9H), 1.99-2.06 (m, 1H), 2.16-2.22 (m, 1H), 3.26-3.32 (m, 1H), 3.44-3.53 (m, 1H), 3.57 (s, 3H), 4.07-4.13 (m, 1H), 6.12 (d, J=7.67 Hz, 1H), 7.50 (d, J=8.59 Hz, 2H), 7.65 (d, J=8.90 Hz, 2H), 7.79 (d, J=8.59 Hz, 2H), 8.03 (d, J=8.90 Hz, 2H), 8.48 (s, 1H); MS (DCI) m/z 449.1 [M+H]⁺.

### Example 57

### (1R,2R)-2-[(4′-{[(cyclohexylamino)carbonyl]amino}-1,1′-biphenyl-4-yl)carbonyl]cyclopentanecarboxylic acid

A scintillation vial was charged with Example 56 (20 mg, 0.04 mmol), LiOH (9 mg, 0.2 mmol) and 6 mL of 4:1 tetrahydrofuran/water. It was placed in a shaker at room temperature overnight. The mixture was concentrated and purified on a RP-HPLC (Preparative reversed-phase chromatography was performed using a Zorbax SB-C18 7 uM 21.2×250 mm column with UV detection analyzed at 220 and 254 nM. Preparative method; (water with 0.1% trifluoroacetic acid and CH₃CN with 0.1% trifluoroacetic acid gradient) 5-95% CH₃CN over 30 minutes at 15 mL/min.) to provide the title product. ¹H NMR (500 MHz, DMSO-d₆) δ ppm 1.14-1.33 (m, 5H), 1.51-1.55 (m, 1H), 1.58-1.73 (m, 4H), 1.73-1.83 (m, 2H), 1.87-1.94 (m, 3H), 1.97-2.05 (m, 1H), 2.95-3.01 (m, 1H), 3.42-3.49 (m, 1H), 4.15-4.21 (m, 1H), 7.17 (br s, 1H), 7.55 (d, J=8.90 Hz, 2H), 7.60 (d, J=8.89 Hz, 2H), 7.76 (d, J=8.59 Hz, 2H), 8.13 (d, J=8.59 Hz, 2H), 9.65 (br s, 1H); MS (ESI) m/z 435.1 [M+H]⁺.

### Example 58

### methyl (1R,2R)-2-[(4′-{[(1-adamantylamino)carbonyl]amino}-1,1′-biphenyl-4-yl)carbonyl]cyclopentanecarboxylate

Example 58 was prepared using the same procedure as described for Example 56, substituting 1-adamantyl isocyanate for cyclohexyl isocyanate. ¹H NMR (500 MHz, DMSO-d6) δ ppm 1.56-1.68 (m, 8H), 1.74-2.06 (m, 12H), 2.18-2.22 (m, 1H), 3.29-3.33 (m, 1H), 3.57 (s, 3H), 4.07-4.12 (m, 1H), 5.94 (s, 1H), 7.47 (d, J=8.54 Hz, 2H), 7.64 (d, J=8.85 Hz, 2H), 7.79

### Example 59

### (1R,2R)-2-[(4′-{[(1-adamantylamino)carbonyl]amino}-1,1′-biphenyl-4-yl)carbonyl]cyclopentanecarboxylic acid

Example 59 was prepared using the procedure as described for Example 57, substituting Example 58 for Example 56. ¹H NMR (500 MHz, DMSO-d₆) δ ppm 1.59-1.66 (m, 8H), 1.68-1.85 (m, 2H), 1.90-1.98 (m, 7H), 2.00-2.10 (m, 3H), 2.11-2.16 (m, 1H), 3.13-3.19 (m, 1H), 4.07-4.13 (m, 1H), 6.08 (s, 1H), 7.48 (d, J=8.90 Hz, 2H), 7.62 (d, J=8.90 Hz, 2H), 7.77 (d, J=8.59 Hz, 2H), 8.05 (d, J=8.28 Hz, 2H), 8.62 (s, 1H); MS (ESI) m/z 487.3 [M+H]⁺.

### Example 60

### methyl (1R,2R)-2-[(4′-{[(cyclopentylamino)carbonyl]amino}-1,1′-biphenyl-4-yl)carbonyl]cyclopentanecarboxylate

Example 60 was prepared using the procedure as described for Example 56 substituting cyclopentyl isocyanate for cyclohexyl isocyanate. ¹H NMR (500 MHz, DMSO-d₆) δ ppm 1.34-1.42 (m, 2H), 1.52-1.70 (m, 6H), 1.73-1.89 (m, 4H), 1.99-2.07 (m, 1H), 2.16-2.22 (m, 1H), 3.28-3.35 (m, 1H), 3.57 (s, 3H), 3.93-3,98 (m, 1H), 4.07-4.14 (m, 1H), 6.21 (d, J=7.05 Hz, 1H), 7.51 (d, J=8.90 Hz, 2H), 7.65 (d, J=8.90 Hz, 2H), 7.79 (d, J=8.59 Hz, 2H), 8.03 (d, J=8.59 Hz, 2H), 8.43 (s, 1H); MS (ESI) m/z 435.2 [M+H]⁺.

### Example 61

### methyl (1R,2R)-2-[(4′-{[(cycloheptylamino)carbonyl]amino}-1,1′-biphenyl-4-yl)carbonyl]cyclopentanecarboxylate

Example 61 was prepared using the procedure as described for Example 56 substituting cycloheptyl isocyanate for cyclohexyl isocyanate. ¹H NMR (500 MHz, DMSO-d₆) δ ppm 1.41-1.87 (m, 16H), 2.00-2.07 (m, 1H), 2.16-2.23 (m, 1H), 3.29-3.32 (m, 1H), 3.57 (s, 3H), 3.66-3.73 (m, 1H), 4.08-4.12 (m, 1H), 6.17 (d, J=7.93 Hz, 1H), 7.51 (d, J=8.85 Hz, 2H), 7.65 (d, J=8.85 Hz, 2H), 7.79 (d, J=8.54 Hz, 2H), 8.03 (d, J=8.55 Hz, 2H), 8.49 (s, 1H); MS (ESI) m/z 463.3 [M+H]⁺.

### Example 62

### methyl (1R,2R)-2-[(4′-{[(1,2,3,4-tetrahydronaphthalen-1-ylamino)carbonyl]amino}-1,1′-biphenyl-4-yl)carbonyl]cyclopentanecarboxylate

Example 62 was prepared using the procedure as described for Example 56 substituting 1-isocyanato-1,2,3,4-tetrahydronaphthalene for cyclohexyl isocyanate, ¹H NMR (500 MHz, DMSO-d₆) δ ppm 1.56-2.06 (m, 9H), 2.11-2.23 (m, 1H), 2.67-2.83 (m, 2H), 3.28-3.35 (m, 1H), 3.58 (s, 3H), 4.06-4.13 (m, 1H), 4.84-4.90 (m, 1H), 6.56 (d, J=8.28 Hz, 1H), 7.10-7.19 (m, 3H), 7.29-7.32 (m, 1H), 7.55 (d, J=8.90 Hz, 2H), 7.68 (d, J]=8.90 Hz, 2H), 7.80 (d, J=8.59 Hz, 2H), 8.04 (d, J=8.29 Hz, 2H), 8.51 (s, 1H); MS (ESI) m/z 497.3 [M+H]⁺.

### Example 63

### (1R,2R)-2-[(4′-{[(cyclopentylamino)carbonyl]amino}-1,1′-biphenyl-4-yl)carbonyl]cyclopentanecarboxylic acid

Example 63 was prepared using the procedure as described for Example 57 substituting Example 60 for Example 56. ¹H NMR (500 MHz, DMSO-d₆) δ ppm 1.34-1.41 (m, 2H), 1.52-1.69 (m, 6H), 1.71-1.88 (m, 4H), 1.99-2.04 (m, 1H), 2.13-2.20 (m, 1H), 3.20-3.24 (m, 1H), 3.94-3.98 (m, 1H), 4.06-4.11 (m, 1H), 6.22 (d, J=7.32 Hz, 1H), 7.51 (d, J=8.85 Hz, 2H), 7.65 (d, J=8.85 Hz, 2H), 7.79 (d, J=8.55 Hz, 2H), 8.04 (d, J=8.54 Hz, 2H), 8.45 (s, 1H), 12.22 (s, 1H); MS (ESI) m/z 421.2 [M+H]⁺.

### Example 64

### (1R,2R)-2-[(4′-{[(cycloheptylamino)carbonyl]amino}-1,1′-biphenyl-4-yl)carbonyl]cyclopentanecarboxylic acid

Example 64 was prepared using the procedure as described for Example 57 substituting Example 61 for Example 56. ¹H NMR (500 MHz, DMSO-d₆) δ ppm 1.43-1.86 (m, 16H), 1.98-2.04 (m, 1H), 2.1-2.20 (m, 1H), 3.20-3.24 (m, 1H), 3.68-3.73 (m, 1H), 4.06-4.11 (m, 1H), 6.17 (d, J=7.93 Hz, 1H), 7.50 (d, J=8.85 Hz, 2H), 7.65 (d, J=8.85 Hz, 2H), 7.79 (d, J=8.54 Hz, 2H), 8.04 (d, J=8.54 Hz, 2H), 8.46 (s, 1H), 12.22 (s, 1H); MS (ESI) m/z 449.2 [M+H]⁺.

### Example 65

### (1R,2R)-2-[(4′-{[(1,2,3,4-tetrahydronaphthalen-1-ylamino)carbonyl]amino}-1,1′-biphenyl-4-yl)carbonyl]cyclopentanecarboxylic acid

Example 65 was prepared using the same procedure as described for Example 57 substituting Example 62 for Example 56. ¹H NMR (500 MHz, DMSO-d₆) δ ppm 1.56-2.02 (m, 9H), 2.11-2.19 (m, 1H), 2.68-2.83 (m, 2H), 3.17-3.21 (m, 1H), 4.07-4.12 (m, 1H), 4.87-4.90 (m, 1H), 6.62-6.69 (m, 1H), 7.10-7.18 (m, 3H), 7.29-7.31 (m, 1H), 7.55 (d, J=8.85 Hz, 2H), 7.68 (d, J=8.84 Hz, 2H), 7.80 (d, J=8.55 Hz, 2H), 8.05 (d, J=8.54 Hz, 2H), 8.61 (s, 1H); MS (ESI) m/z 483.2 [M+H]⁺.

### Example 66

### methyl (1R,2R)-2-[(4′-{[(cyclooctylamino)carbonyl]amino}-1,1′-biphenyl-4-yl)carbonyl]cyclopentanecarboxylate

Example 66 was prepared using the procedure as described for Example 56 substituting cyclooctyl isocyanate for cyclohexyl isocyanate. ¹H NMR (500 MHz, DMSO-d₆) δ ppm 1.42-1.86 (m, 18H), 1.99-2.06 (m, 1H), 2.15-2.22 (m, 1H), 3.15-3.24 (m, 1H), 3.26-3.24 (m, 1H), 3.57 (s, 3H), 4.07-4.13 (m, 1H), 6.22 (d, J=7.98 Hz, 1H), 7.51 (d, J=8.59 Hz, 2H), 7.65 (d, J=8.59 Hz, 2H), 7.79 (d, J=8.90 Hz, 2H), 8.03 (d, J=8.59 Hz, 2H), 8.51 (s, 1H); MS (ESI) m/z 477.3 [M+H]⁺.

### Example 67

### (1R,2R)-2-[(4′-{[(cyclooctylamino)carbonyl]amino}-1,1′-biphenyl-4-yl)carbonyl]cyclopentanecarboxylic acid

Example 67 was prepared using the same procedure as described for Example 57 substituting Example 66 for Example 56. ¹H NMR (500 MHz,D MSO-d₆) δ ppm 1.42-1.86 (m, 18H), 1.98-2.04 (m, 1H), 2.13-2.19 (m, 1H), 3.16-3.24 (m, 1H), 3.68-3.73 (m, 1H), 4.06-4.10 (m, 1H), 6.18 (d, J=7.94 Hz, 1H), 7.50 (d, J=8.55 Hz, 2H), 7.65 (d, J=8.85 Hz, 2H), 7.79 (d, J=8.55 HZ, 2H), 8.04 (d, J=8.55 Hz, 2H), 8.46 (s, 1H), 12.22 (s, 1H); MS (ESI) m/z 463.3 [M+H]⁺.

It is understood that the foregoing detailed description and accompanying examples are merely illustrative and are not to be taken as limitations upon the scope of the invention, which is defined solely by the appended claims and their equivalents. Various changes and modifications including, hut not limited to, those relating to the chemical structures, substituents, derivatives, intermediates, syntheses, formulations and/or methods of use of the invention, can be made without departing from the spirit and scope thereof.

We claim:

1. A compound of formula (I) 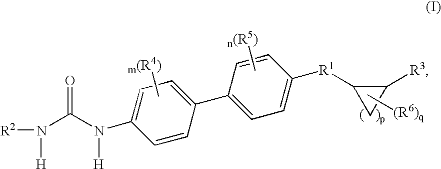
or a pharmaceutically acceptable salt, prodrug, salt of a prodrug, or a combination thereof, wherein
R¹ is C(O) or C(H)OH;
R² is alkyl, aryl, heteroaryl or cycloalkyl; wherein each of the aryl, heteroaryl and cycloalkyl is independently unsubstituted or substituted with 1, 2, 3, 4, or 5 substituents independently selected from the group consisting of alkyl, alkenyl, alkynyl, nitro, —CN, halogen, ethylenedioxy, methylenedioxy, haloalkyl, —OR^{a}, —O—C(O)(R^{a}), —S(R^{a}), —S(O)(R^{b}), —S(O)₂(R^{b}), —C(O)(R^{a}), —C(O)(OR^{a}), —N(R^{a})₂, —(R^{a})—C(O)(R^{a}), —C(O)N(R^{a})₂, —S(O)₂N(R^{a})₂, R⁷, —(CR^{c}R^{d})ₜ—OR^{a}, —(CR^{c}R^{d})ₜ—O—C(O)(R^{a}), —(CR^{c}R^{d})ₜ—S(R^{a}), —(CR^{c}R^{d})ₜ—S(O)(R^{b}), —(CR^{c}R^{d})ₜ—S(O)₂(R^{b}), —(CR^{c}R^{d})ₜ—C(O)(R^{a}), —(CR^{c}R^{d})ₜ—C(O)(OR^{a}), —(CR^{c}R^{d})ₜ—N(R^{a})₂, —(CR^{c}R^{d})ₜ—N(R^{a})—C(O)(R^{a}), —(CR^{c}R^{d})ₜ—C(O)N(R^{a})₂, —(CR^{c}R^{d})ₜ—S(O)₂N(R^{a})₂ and —(CR^{c}R^{d})ₜ—R⁷;
R³, at each occurrence, is independently —C(O)O(R⁸) or —C(O)N(R⁸)₂;
R⁴ and R⁵ represent substituent groups independently selected from the group consisting of alkyl, alkenyl, alkynyl, nitro, —CN, halogen, haloalkyl, —OR^{e}, —O—C(O)(R^{e}), —S(R^{e}), —S(O)(R^{f}), —S(O)₂(R^{f}), —C(O)(R^{e}), —C(O)(OR^{e}), —N(R^{e})₂, —N(R^{e})—C(O)(R^{e}), —C(O)N(R^{e})₂, —S(O)₂N(R^{e})₂, —(CR^{c}R^{d})ₜ—OR^{e}, —(CR^{c}R^{d})ₜ—O—C(O)(R^{e}), —(CR^{c}R^{d})ₜ—S(R^{e}), —(CR^{c}R^{d})ₜ—S(O)(R^{f}), —(CR^{c}R^{d})ₜ—S(O)₂(R^{f}), —(CR^{c}R^{d})ₜ—C(O)(R^{e}), —(CR^{c}R^{d})ₜ—C(O)(OR^{e}), —(CR^{c}R^{d})ₜ—N(R^{e})₂, —(CR^{c}R^{d})ₜ—N(R^{e})—C(O)(R^{e}), —(CR^{c}R^{d})ₜ—C(O)N(R^{e})₂, and —(CR^{c}R^{d})ₜ—S(O)₂N(R^{e})₂;
R⁶ represents a substituent group selected from the group consisting of alkyl, OR^{a} and halogen;
R⁷, at each occurrence, is independently aryl, heteroaryl, cycloalkyl, cycloalkenyl or heterocycle; wherein each R⁷ is independently unsubstituted or substituted with 1, 2, 3, 4 or 5 substituents selected from the group consisting of alkyl, alkenyl, alkynyl, nitro, —CN, halogen, ethylenedioxy, methylenedioxy, haloalkyl, —OR^{e}, —O—C(O)(R^{e}), —S(R^{e}), —S(O)(R^{f}), —S(O)₂(R^{f}), —C(O)(R^{e}), —C(O)(OR^{e}), —N(R^{e})₂, —N(R^{e})—C(O)(R^{e}), —C(O)N(R^{e})₂, —S(O)₂N(R^{e})₂, —(CR^{c}R^{d})ₜ—OR^{e}, —(CR^{c}R^{d})ₜ—O—C(O)(R^{e}), —(CR^{c}R^{d})ₜ—S(R^{e}), —(CR^{c}R^{d})ₜ—S(O)(R^{f}), —(CR^{c}R^{d})ₜ—S(O)₂(R^{f}), —(CR^{c}R^{d})ₜ—C(O)(R^{e}), —(CR^{c}R^{d})ₜ—C(O)(OR^{e}), —(CR^{c}R^{d})ₜ—N(R^{e})₂, —(CR^{c}R^{d})ₜ—N(R^{e})—C(O)(R^{e}), —(CR^{c}R^{d})ₜ—C(O)N(R^{e})₂, and —(CR^{c}R^{d})ₜ—S(O)₂N(R^{e})₂;
R^{8,} at each occurrence, is independently hydrogen or alkyl;
R^{a}, at each occurrence, is independently hydrogen, alkyl, haloalkyl, R⁷, or —(CR^{c}R^{d})ₜ—R⁷;
R^{b}, at each occurrence, is independently alkyl, haloalkyl, R⁷, or —(CR^{c}R^{d})ₜ—R⁷;
R^{c}, R^{d} and R^{e}, at each occurrence, are each independently hydrogen, alkyl or haloalkyl;
R^{f}, at each occurrence, is independently alkyl or haloalkyl;
t is 1, 2, 3 or 4;
m is 0, 1, 2 or 3;
n is 0, 1, 2 or 3;
p is 1, 2, 3, 4 or 5; and
q is 0, 1 or 2

2. The compound of claim 1 having formula (Ia) or a pharmaceutically acceptable salt, prodrug, salt of a prodrug, or a combination thereof, 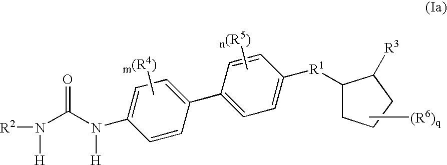
wherein R¹, R², R³, R⁴, R⁵, R⁶, m, n, and q are as defined in claim 1.

3. The compound of claim 2 having formula (Ia) or a pharmaceutically acceptable salt, prodrug, salt of a prodrug, or a combination thereof, wherein R¹ is C(O), R² is alkyl, and R³, R⁴, R⁵, R⁶, m, n, and q are as defined in claim 1.

4. The compound of claim 2 having formula (Ia) or a pharmaceutically acceptable salt, prodrug, salt of a prodrug, or a combination thereof, wherein R¹ is C(O), R² is aryl, and R³, R⁴, R⁵, R⁶, m, n, and q are as defined in claim 1.

5. The compound of claim 2 having formula (Ia) or a pharmaceutically acceptable salt, prodrug, salt of a prodrug, or a combination thereof, wherein R¹ is C(O), R² is heteroaryl, and R³, R⁴, R⁵, R⁶, m, n, and q are as defined in claim 1.

6. The compound of claim 2 having formula (Ia) or a pharmaceutically acceptable salt, prodrug, salt of a prodrug, or a combination thereof, wherein R¹ is C(O), R² is cycloalkyl, and R³, R⁴, R⁵, R⁶, m, n, and q are as defined in claim 1.

7. The compound of claim 2 having formula (Ia) or a pharmaceutically acceptable salt, prodrug, salt of a prodrug, or a combination thereof, wherein R¹ is C(H)(OH), R² is aryl, and R³, R⁴, R⁵, R⁶, m, n, and q are as defined in claim 1.

8. The compound of claim 1 having formula (Ib) or a pharmaceutically acceptable salt, prodrug, salt of a prodrug, or a combination thereof, 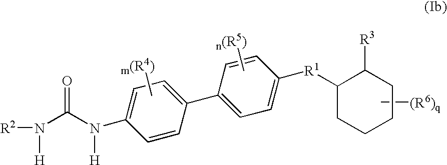
wherein R¹, R², R³, R⁴, R⁵, R⁶, m, n, and q are as defined in claim 1.

9. The compound of claim 3 wherein R¹ is C(O), R² is aryl, and R³, R⁴, R⁵, R⁶, m, n, and q are as defined in claim 1.

10. The compound of claim 1 having formula (I) selected from the group consisting of
methyl (1R,2R)-2-({4′-[({[4-(trifluoromethoxy)phenyl]amino}carbonyl)amino]-1,1′-biphenyl-4-yl}carbonyl)cyclopentanecarboxylate;
(1R,2R)-2-({4′-[({[4-(trifluoromethoxy)phenyl]amino}carbonyl)amino]-1,1′-biphenyl-4-yl}carbonyl)cyclopentanecarboxylic acid;
methyl (1R,2R)-2-({4′-[({[4-(trifluoromethyl)phenyl]amino}carbonyl)amino]-1,1′-biphenyl-4-yl}carbonyl)cyclopentanecarboxylate;
(1R,2R)-2-({4′-[({[4-(trifluoromethyl)phenyl]amino}carbonyl)amino]-1,1′-biphenyl-4-yl}carbonyl)cyclopentanecarboxylic acid;
methyl (1R,2R)-2-[(4′-{[(1,3-benzodioxol-5-ylamino)carbonyl]amino}-1,1′-biphenyl-4-yl)carbonyl]cyclopentanecarboxylate;
(1R,2R)-2-[(4′-{[(1,3-benzodioxol-5-ylamino)carbonyl]amino}-1,1′-biphenyl-4-yl)carbonyl]cyclopentanecarboxylic acid;
methyl (1R,2R)-2-{[4′-({[(4-acetylphenyl)amino]carbonyl}amino)-1,1′-biphenyl-4-yl]carbonyl}cyclopentanecarboxylate;
(1R,2R)-2-{[4′-({[(4-acetylphenyl)amino]carbonyl}amino)-1,1′-biphenyl-4-yl]carbonyl}cyclopentanecarboxylic acid;
methyl (1R,2R)-2-({4′-[(anilinocarbonyl)amino]-1,1′-biphenyl-4-yl}carbonyl)cyclopentanecarboxylate;
(1R,2R)-2-({4′-[(anilinocarbonyl)amino]-1,1′-biphenyl-4-yl}carbonyl)cyclopentanecarboxylic acid;
methyl (1R,2R)-2-{[4′-({[(2-chlorophenyl)amino]carbonyl}amino)-1,1′-biphenyl-4-yl]carbonyl}cyclopentanecarboxylate;
(1R,2R)-2-{[4′-({[(2-chlorophenyl)amino]carbonyl}amino)-1,1′-biphenyl-4-yl]carbonyl}cyclopentanecarboxylic acid;
methyl (1R,2R)-2-{[4′-({[(3-chlorophenyl)amino]carbonyl}amino)-1,1′-biphenyl-4-yl]carbonyl}cyclopentanecarboxylate;
(1R,2R)-2-{[4′-({[(3-chlorophenyl)amino]carbonyl}amino)-1,1′-biphenyl-4-yl]carbonyl}cyclopentanecarboxylic acid;
methyl (1R,2R)-2-{[4′-({[(4-chlorophenyl)amino]carbonyl}amino)-1,1′-biphenyl-4-yl]carbonyl}cyclopentanecarboxylate;
(1R,2R)-2-{[4′-({[(4-chlorophenyl)amino]carbonyl}amino)-1,1′-biphenyl-4-yl]carbonyl}cyclopentanecarboxylic acid;
methyl (1R,2R)-2-{[4′-({[(4-cyanophenyl)amino]carbonyl}amino)-1,1′-biphenyl-4-yl]carbonyl}cyclopentanecarboxylate;
(1R,2R)-2-{[4′-({[(4-cyanophenyl)amino]carbonyl}amino)-1,1′-biphenyl-4-yl]carbonyl}cyclopentanecarboxylic acid;
methyl (1R,2R)-2-{[4′-({[(4-methoxyphenyl)amino]carbonyl}amino)-1,1′-biphenyl-4-yl]carbonyl}cyclopentanecarboxylate;
(1R,2R)-2-{[4′-({[(4-methoxyphenyl)amino]carbonyl}amino)-1,1′-biphenyl-4-yl]carbonyl}cyclopentanecarboxylic acid;
methyl (1R,2R)-2-[(4′-{[(isopropylamino)carbonyl]amino}-1,1′-biphenyl-4-yl)carbonyl]cyclopentanecarboxylate;
(1R,2R)-2-[(4′-{[(isopropylamino)carbonyl]amino}-1,1′-biphenyl-4-yl)carbonyl]cyclopentanecarboxylic acid;
methyl 2-({4′-[({[4-(trifluoromethyl)phenyl]amino}carbonyl)amino]-1,1′-biphenyl-4-yl}carbonyl)cyclohexanecarboxylate;
2-({4′-[({[4-(trifluoromethyl)phenyl]amino}carbonyl)amino]-1,1′-biphenyl-4-yl}carbonyl)cyclohexanecarboxylic acid;
methyl 2-{[4′-({([(3-chlorophenyl)amino]carbonyl}amino)-1,1′-biphenyl-4-yl]carbonyl}cyclohexanecarboxylate;
2-{[4′-({[(3-chlorophenyl)amino]carbonyl}amino)-1,1′-biphenyl-4-yl]carbonyl}cyclohexanecarboxylic acid;
(1R,2R)-2-((R)-hydroxy {4′-[({[4-(trifluoromethyl)phenyl]amino}carbonyl)amino]-1,1′-biphenyl-4-yl}methyl)cyclopentanecarboxylic acid;
(1R,2R)-2-((S)-hydroxy{4′-[({[4-(trifluoromethyl)phenyl]amino}carbonyl)amino]-1,1′-biphenyl-4-yl}methyl)cyclopentanecarboxylic acid;
methyl (1R,2R)-2-{[4′-({[(4-fluorophenyl)amino]carbonyl}amino)-1,1′-biphenyl-4-yl]carbonyl}cyclopentanecarboxylate;
(1R,2R)-2-{[4′-({[(4-fluorophenyl)amino]carbonyl}amino)-1,1′-biphenyl-4-yl]carbonyl}cyclopentanecarboxylic acid;
methyl (1R,2R)-2-{[4′-({[(3-fluorophenyl)amino]carbonyl}amino)-1,1′-biphenyl-4-yl]carbonyl}cyclopentanecarboxylate;
(1R,2R)-2-{[4′-({[(3-fluorophenyl)amino]carbonyl}amino)-1,1′-biphenyl-4-yl]carbonyl}cyclopentanecarboxylic acid;
methyl (1R,2R)-2-{[4′-({[(2-fluorophenyl)amino]carbonyl}amino)-1,1′-biphenyl-4-yl]carbonyl}cyclopentanecarboxylate;
(1R,2R)-2-{[4′-({[(2-fluorophenyl)amino]carbonyl}amino)-1,1′-biphenyl-4-yl]carbonyl}cyclopentanecarboxylic acid;
methyl 2-{[4′-({[(4-fluorophenyl)amino]carbonyl}amino)-1,1′-biphenyl-4-yl]carbonyl}cyclohexanecarboxylate;
2-{[4′-({[(4-fluorophenyl)amino]carbonyl}amino)-1,1′-biphenyl-4-yl]carbonyl}cyclohexanecarboxylic acid;
methyl (1R,2R)-2-({4′-[({[3-(trifluoromethyl)phenyl]amino}carbonyl)amino]-1,1′-biphenyl-4-yl}carbonyl)cyclopentanecarboxylate;
(1R,2R)-2-({4′-[({[3-(trifluoromethyl)phenyl]amino]carbonyl)amino]-1,1′-biphenyl-4-yl}carbonyl)cyclopentanecarboxylic acid;
methyl 2-({4′-[({[3-(trifluoromethyl)phenyl]amino}carbonyl)amino]-1,1′-biphenyl-4-yl}carbonyl)cyclohexanecarboxylate; and
2-({4′-[({[3-(trifluoromethyl)phenyl]amino}carbonyl)amino]-1,1′-biphenyl-4-yl}carbonyl)cyclohexanecarboxylic acid;
methyl (1R,2R)-2-({4′-[({[2-(trifluoromethyl)phenyl]amino}carbonyl)amino]-1,1′-biphenyl-4-yl}carbonyl)cyclopentanecarboxylate;
(1R,2R)-2-({4′-[({[2-(trifluoromethyl)phenyl]amino}carbonyl)amino]-1,1′-biphenyl-4-yl}carbonyl)cyclopentanecarboxylic acid;
methyl 2-({4′-[({[2-(trifluoromethyl)phenyl]amino}carbonyl)amino]-1,1′-biphenyl-4-yl}carbonyl)cyclohexanecarboxylate 2-({4′-[({[2-(trifluoromethyl)phenyl]amino}carbonyl)amino]-1,1′-biphenyl-4-yl}carbonyl)cyclohexanecarboxylic acid;
(1R,2R)-2-({4′-[(anilinocarbonyl)amino]-1,1′-biphenyl-4-yl}carbonyl)cyclopentanecarboxamide;
(1R,2R)-2-({4′-[(anilinocarbonyl)amino]-1,1′-biphenyl-4-yl}carbonyl)-N-methylcyclopentanecarboxamide;
methyl (1R,2R)-2-{[4′-({[(2,6-diisopropylphenyl)amino]carbonyl}amino)-1,1′-biphenyl-4-yl]carbonyl}cyclopentanecarboxylate;
(1R,2R)-2-{[4′-({[(2,6-diisopropylphenyl)amino]carbonyl}amino)-1,1′-biphenyl-4yl]carbonyl}cyclopentanecarboxylic acid;
methyl (1R,2R)-2-{[4′-({[(2,6-dimethylphenyl)amino]carbonyl}amino)-1,1′-biphenyl-4-yl]carbonyl}cyclopentanecarboxylate;
(1R,2R)-2-{[4′-({[(2,6-dimethylphenyl)amino]carbonyl}amino)-1,1′-biphenyl-4-yl]carbonyl]cyclopentanecarboxylic acid;
methyl (1R,2R)-2-({4′-[({[2-fluoro-5-(trifluoromethyl)phenyl]amino}carbonyl)amino]-1,1′-biphenyl-4-yl}carbonyl)cyclopentanecarboxylate;
(1R,2R)-2-({4′-[({[2-fluoro-5-(trifluoromethyl)phenyl]amino}carbonyl)amino]-1,1′-biphenyl-4-yl}carbonyl)cyclopentanecarboxylic acid;
methyl (1R,2R)-2-[(4′-{[(thien-2-ylamino)carbonyl]amino}-1,1′-biphenyl-4-yl)carbonyl]cyclopentanecarboxylate;
(1R,2R)-2-[(4′-{[(thien-2-ylamino)carbonyl]amino}-1,1′-biphenyl-4-yl)carbonyl]cyclopentanecarboxylic acid;
methyl (1R,2R)-2-[(4′-{[(pyridin-3-ylamino)carbonyl]amino)-1,1′-biphenyl-4-yl)carbonyl]cyclopentanecarboxylate;
(1R,2R)-2-[(4′-{[(pyridin-3-ylamino)carbonyl)amino}-1,1′-biphenyl-4-yl)carbonyl]cyclopentanecarboxylic acid;
methyl (1R,2R)-2-[(4′-{[(cyclohexylamino)carbonyl]amino}-1,1′-biphenyl-4-yl)carbonyl]cyclopentanecarboxylate;
(1R,2R)-2-[(4′-{[(cyclohexylamino)carbonyl]amino}-1,1′-biphenyl-4-yl)carbonyl]cyclopentanecarboxylic acid;
methyl (1R,2R)-2-[(4′-{[(1-adamantylamino)carbonyl]amino}-1,1′-biphenyl-4-yl)carbonyl]cyclopentanecarboxylate;
(1R,2R)-2-[(4′-{[(1-adamantylamino)carbonyl]amino}-1,1′-biphenyl-4-yl)carbonyl]cyclopentanecarboxylic acid;
methyl (1R,2R)-2-[(4′-{[(cyclopentylamino)carbonyl]amino}-1,1′-biphenyl-4-yl)carbonyl]cyclopentanecarboxylate;
methyl (1R,2R)-2-[(4′-{[(cycloheptylamino)carbonyl]amino}-1,1′-biphenyl-4-yl)carbonyl]cyclopentanecarboxylate;
methyl (1R,2R)-2-[(4′-{[(1,2,3,4-tetrahydronaphthalen-1-ylamino)carbonyl]amino}-1,1′-biphenyl-4-yl)carbonyl]cyclopentanecarboxylate;
(1R,2R)-2-[(4′-{[(cyclopentylamino)carbonyl]amino}-1,1′-biphenyl-4-yl)carbonyl]cyclopentanecarboxylic acid;
(1R,2R)-2-[(4′-{[(cycloheptylamino)carbonyl]amino}-1,1′-biphenyl-4-yl)carbonyl]cyclopentanecarboxylic acid;
(1R,2R)-2-[(4′-{[(1,2,3,4-tetrahydronaphthalen-1-ylamino)carbonyl]amino}-1,1′-biphenyl-4-yl)carbonyl]cyclopentanecarboxylic acid;
methyl (1R,2R)-2-[(4′-{[(cyclooctylamino)carbonyl]amino}-1,1′-biphenyl-4-yl)carbonyl]cyclopentanecarboxylate; and
(1R,2R)-2-[(4′-{[(cyclooctylamino)carbonyl]amino}-1,1′-biphenyl-4-yl)carbonyl]cyclopentanecarboxylic acid;
or a pharmaceutically acceptable salt, prodrug, or salt of a prodrug thereof.

11. A pharmaceutical composition comprising a therapeutically effective amount of a compound of claim 1 in combination with a pharmaceutically acceptable carrier.

12. A method for treating disorders by inhibiting DGAT-1 comprising the step of administering to a subject in need thereof a therapeutically effective amount of a compound of claim 1.

13. A method for treating type II diabetes comprising the step of administering to a subject in need thereof a therapeutically effective amount of a compound of claim 1.

14. A method for treating non-alcoholic fatty liver disease comprising the step of administering to a subject in need thereof a therapeutically effective amount of a compound of claim 1.

15. A method for lowering plasma triacylglycerides comprising the step of administering to a subject in need thereof a therapeutically effective amount of a compound of claim 1.

16. The method of claim 15 further comprising the step of co-administering with a therapeutically effective amount of fenofibrate.

17. A method for treating non-alcoholic steatohepatitis comprising the step of administering to a subject in need thereof a therapeutically effective amount of a compound of claim 1.

18. A method for treating obesity comprising the step of administering to a subject in need thereof a therapeutically effective amount of a compound of claim 1.

19. A method for treating obesity comprising the step of administering to a subject in need thereof a therapeutically effective amount of a compound of claim 1, in combination with one or more pharmaceutical agents selected from the group consisting of fenofibrate and Rimonabant.

20. A pharmaceutical composition comprising a compound of claim 1, a pharmaceutical agent selected from the group consisting of fenofibrate and Rimonabant, and a pharmaceutically acceptable carrier.
